(19) United States
(12) Patent Application Publication
(10) Pub. No.: US 2003/0026806 A1
(43) Pub. Date: Feb. 6, 2003

(54) Antibodies and other selective IL-1 binding agents that allow binding to IL-1 receptor but not activation thereof

(75) Inventors: Alison Witte, Camarillo, CA (US); Brian C. Varnum, Santa Monica, CA (US); Xueming Qian, Oak Park, CA (US); Chris Vezina, Newbury Park, CA (US)

Correspondence Address: U.S. Patent Operations/ TJG, Dept. 4300, M/S 27-4-A, AMGEN INC., One Amgen Center Drive, Thousand Oaks, CA, 91320-1799, US

(73) Assignee: Amgen Inc.

(21) Appl. No.: 10/011,931

(22) Filed: Oct. 29, 2001

Related U.S. Application Data
(60) Provisional application No. 60/244,118, filed on Oct. 27, 2000.

(51) Int. Cl. A61K 39/395; C12N 15/86; C12P 21/02

(52) U.S. Cl. 424/145.1; 435/69.1; 435/456

(57) ABSTRACT

The invention concerns methods of treating IL-1-mediated disease by administering a selective IL-1 binding agents that form an antagonist complex with the IL-1 ligand. The antagonist complex can bind to the IL-1 receptor without activating the receptor. The selective binding agents contemplated by this invention thus have the properties of free ligand absorption (through binding of the selective binding agent to free IL-1α or β) and receptor activation inhibition (through binding of the antagonist complex to the receptor and preventing its activation).

This application claims the benefit of U.S. Provisional Application No. 60/244,118, filed Oct. 27, 2000, which is hereby incorporated by reference.

### FIELD OF THE INVENTION

This invention is in the field of pharmaceutical agents and specifically relates to antibodies and other selective binding agents, compositions, uses and methods for treating rheumatoid arthritis and other IL-1 mediated disorders.

### BACKGROUND OF THE INVENTION

One of the most potent inflammatory cytokines yet discovered is interleukin-1 (IL-1). IL-1 is thought to be a key mediator in many diseases and medical conditions. It is manufactured primarily (though not exclusively) by cells of the macrophage/monocyte lineage and may be produced in two forms: IL-1 alpha (IL-1α) and IL-1 beta (IL-1β).

IL-1 stimulates cellular responses by interacting with a heterodimeric receptor complex comprised of two transmembrane proteins, IL-1 receptor type I (IL-1R1) and IL-1 receptor accessory protein (IL-1RAcP). IL-1 first binds to IL-1R1. IL-1RAcP is then recruited to this complex. Greenfeder et al. (1995), J. Biol. Chem 270:13757-65; Yoon & Dinarello (1998), J. Immunology 160:3170-79; Cullinan et al. (1998), J. Immunology 161:5614-20. Cell-based binding studies suggest that IL-1RAcP stabilizes the IL-1R1 signaling complex by slowing the ligand off-rate (Wesche et al. (1998), FEBS Letters 429:303-306).

While the stoichiometry of the IL-1 binding to IL-1R1 has been thoroughly characterized, the interaction of IL-1RAcP with ligand-bound receptor remains poorly defined. Since IL-1RAcP has no significant affinity for either IL-1 or IL-1R1 alone, it is likely that novel binding sites for IL-1RAcP are created by the initial IL-1/IL-1R1 binding event (Ettorre et al. (1997), Eur. Cytokine Netw. 8(2):161-171).

Antibodies to IL-1β have been previously reported. See U.S. Pat. Nos. 4,935,343; 5,348,858; and 5,681,933. These antibodies generally bound to sites on IL-1 that are involved in binding to IL-1 receptor or are silent on the mechanism of their inhibition of IL-1 mediated signaling. See col. 3, lines 1 to 49 of U.S. Pat. No. 4,935,343; col. 3, lines 62 to 68 of U.S. Pat. No. 5,348, 858.

IL-1β antibodies are one of several classes of IL-1 inhibitors that have been proposed, including:

Antagonists for binding to IL-1R1, based on naturally occurring molecules such as IL-1ra. Receptor antagonists (including IL-1ra and variants and derivatives thereof), as well as methods of making and using thereof, are described in U.S. Pat. No. 5,075,222; WO 91/08285; WO 91/17184; AU 9173636; WO 92/16221; WO93/21946; WO 94/06457; WO 94/21275; FR 2706772; WO 94/21235; DE 4219626, WO 94/20517; WO 96/22793;WO 97/28828; and WO 99/36541.

Antibodies to IL-1R1, which would bind to the receptor and prevent binding by IL-1.

IL-1 binding proteins based on naturally occurring molecules such as soluble IL-1 receptors. Exemplary IL-1 binding proteins are described in U.S. Pat. Nos. 5,492,888; 5,488,032; 5,464,937; 5,319,071; and 5,180,812.

Antibodies to IL-1. Exemplary antibodies are described in WO 9501997, WO 9402627, WO 9006371, U.S. Pat. No. 4,935,343, EP 364778, EP 267611 and EP 220063, the disclosures of which are hereby incorporated by reference.

Antibodies to IL-1RAcP (see e.g., WO 96/23067 and WO 99/37773).

Inhibitors of IL-1β converting enzyme (ICE) or caspase I (e.g., WO 99/46248, WO 99/47545, and WO 99/47154), which can be used to inhibit IL-1β production and secretion;

IL-1β protease inhibitors.

The mechanism of action of the foregoing inhibitors can be classified as either (a) “upstream inhibition”—reduced production or release of IL-1 (ICE inhibitors, IL-1β protease inhibitors); (b) “free ligand absorption”—binding to free IL-1 and thus preventing its binding to IL-1R (soluble IL-1 receptors, IL-1 antibodies); (c) “receptor antagonism”—binding to IL-1R1 and competitively inhibiting free IL-1 from binding to IL-1R1 (IL-1ra, IL-1R antibodies); and (d) “receptor activation inhibition”—preventing activation of the IL-1 receptor (antibodies to IL-1 receptor accessory protein). The art would benefit from inhibitors that combine two or more of these properties.

### SUMMARY OF THE INVENTION

The present invention concerns a method of treating IL-1-mediated disease, which comprises administering a selective binding agent for IL-1β, wherein the resulting selective binding agent-IL-1β complex (“antagonist complex” as defined hereinafter) is capable of binding to the IL-1 receptor without activating the receptor. Conversely, the invention also concerns administering a selective binding agent for IL-1α that also forms such an antagonist complex. The selective binding agents contemplated by this invention thus have the properties of free ligand absorption (through binding of the selective binding agent to free IL-1α or β) and receptor activation inhibition (through binding of the antagonist complex to the receptor and preventing its activation).

This invention thus concerns a method of treatment wherein the antagonist complex is capable of blocking association of the IL-1 receptor with IL-1 receptor accessory protein.

Further, the invention concerns a method of blocking both IL-1α and IL-1β from binding to the IL-1 receptor, which comprises administering a selective binding agent for IL-1β, wherein the resulting selective binding agent-IL-1 complex is capable of binding to IL-1 receptor without activating said receptor. Conversely, this invention concerns a method of blocking IL-1α and IL-1β from binding to the IL-1 receptor, which comprises administering an IL-1 selective binding agent for IL-1α, wherein the resulting selective binding agent-IL-1 complex is capable of binding to IL-1 receptor without activating said receptor.

The selective binding agent used in this invention is preferably an antibody or an antibody fragment. Such antibody is preferred to be a fully human antibody and may be generated by phage display techniques. Chimeric and humanized antibodies may also be used. Alternatively, the selective binding agent may be a peptide or a fusion protein comprising a peptide. Peptides and exemplary fusion proteins comprising peptides are described in an international patent application entitled, “Modified Peptides as Thearpeutic Agents,” WO 99/25044.

### BRIEF DESCRIPTION OF THE FIGURES

FIG. 1 shows the structure of recombinant human IL-1RAcP constructs. Native IL-1RAcP consists of an extracellular portion containing three Ig-like domains, a short transmembrane domain and an intracellular domain involved in IL-1β signal transduction (FIG. 1, A). A soluble, chimeric protein consisting of the complete extracellular domain of IL-1RAcP joined at its c-terminus to human Fc was assembled in a mammalian expression vector (FIG. 1, B).

FIG. 2 shows the results from a synoviocyte assay as described below. This figure shows that IL-1β-dependent IL-6 production in SW982 is blocked by inhibitors targeting each protein of the IL-1β signaling complex. Anti-IL-1 antibody MAB201 is the most effective inhibitor of IL-1β signaling.

FIG. 3 shows the results of a HUVEC assay as described below. In this assay, IL-1β-dependent GROα production in HUVEC is blocked by inhibitors targeting each of the IL-1β signaling complex. Anti-IL-1β antibody MAB201 is the most effective inhibitor of IL-1β signaling.

FIG. 4 shows in vitro binding assays with recombinant proteins, demonstrating two distinct mechanisms of action for biologically neutralizing anti-IL-1β antibodies. AF201 blocks binding of IL-1β to sIL-1R1 whereas MAB201 does not (FIG. 4A). Both antibodies inhibit IL-1RAcP binding to IL-1 bound IL-1R1 (FIG. 4B).

FIG. 5 is a schematic model showing that the activation of the IL-1 signaling pathway starts with the binding of IL-1β to type I IL-1 receptor, followed by the recruitment of IL-1RacP into the complex (I).

FIGS. 6A and 6B shows the alignment of murine light chain (FIG. 6a) and murine heavy chain (FIG. 6b) variable regions with human germline acceptor sequence.

FIGS. 7A and 7B show a comparison of activity of MAb201-chimera and MAb201.

FIG. 7A shows a dose-response curve of IL-1-induced IL-6 in primary human chondrocytes (P4, 20 hr).

FIG. 7B shows a dose-dependent inhibition of IL-1β-induced IL-6 in primary human chondrocytes by MAb201 and MAb201-chimera (1 pM, P4, 20 hr). FIG. 7B leads to the conclusion that MAb201 and MAb201-chimera have identical activity.

FIG. 8 shows the relative potency of IL-1ra, MAb201, MAb201-Chimera in human chondrocytes. Human chondrocytes were stimulated with IL-1β or IL-1β in the presence of 1 nM IL-1 inhibitor and IL-6 in cell culture supernatant was measured after 16 hour stimulation using ELISA. Data were analyzed using Prism Graphpad. FIG. 8 leads to the conclusion that MAb201-CDR is as potent as IL-1ra in inhibiting IL-1 induced IL-6 in human chondrocytes.

FIG. 9 shows IL-1-induced PGE2 production in human fibroblast-like synoviocytes (P5, 1 pM IL-1β). These data show the relative potency of different classes of IL-1 inhibitors: anti-IL-1β (MAb201) and IL-1ra in human fibroblast-like synoviocytes. Human fibroblast-like synoviocytes were stimulated with IL-1β or IL-10 in the presence of 1 nM IL-1 inhibitor and IL-6 in cell culture supernatant was measured after 16 hours stimulation using ELISA. Data were analyzed using Prism Graphpad.

FIGS. 10A and 10B show the results of BiaCore analysis. The purified CDR-grafted MAb201 (FIG. 10A) and chimeric MAb201 (FIG. 10B) were tested in a direct binding analysis on BlAcore. Various amounts of IL-1β was injected over the protein G-captured MAb201 surface. The binding affinity was estimated for the chimeric and the CDR-grafted antibody at sub-nanomole with very slow off rates.

FIG. 11 shows the activities of mouse, chimeric and humanized MAB201 in blocking IL-1β driven IL-1RacP binding to IL-1 bound IL-1R1. Varying concentrations of each antibody were incubated with biotinylated soluble IL-1 receptor and ruthenium-tagged IL-1 receptor accessory protein (IL-1RacP). The resulting complexes were captured using magnetic streptavidin-coated beads and an Origen® Analyzer (IGEN Inc.) measured an ECL signal proportional to the ruthenium tag captured in the complex to evaluate competition of complex formation.

### DETAILED DESCRIPTION OF THE INVENTION

In General

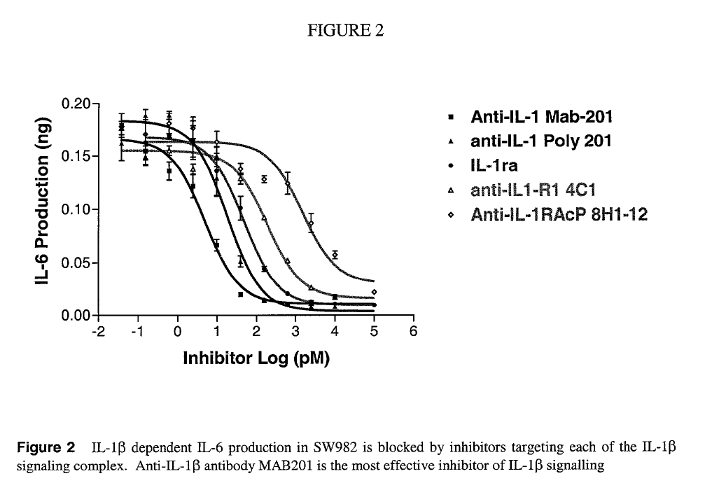

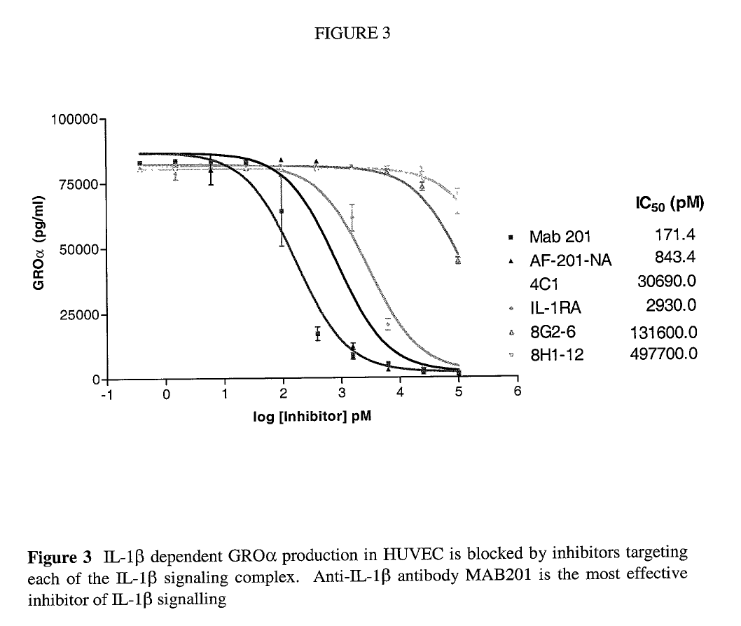

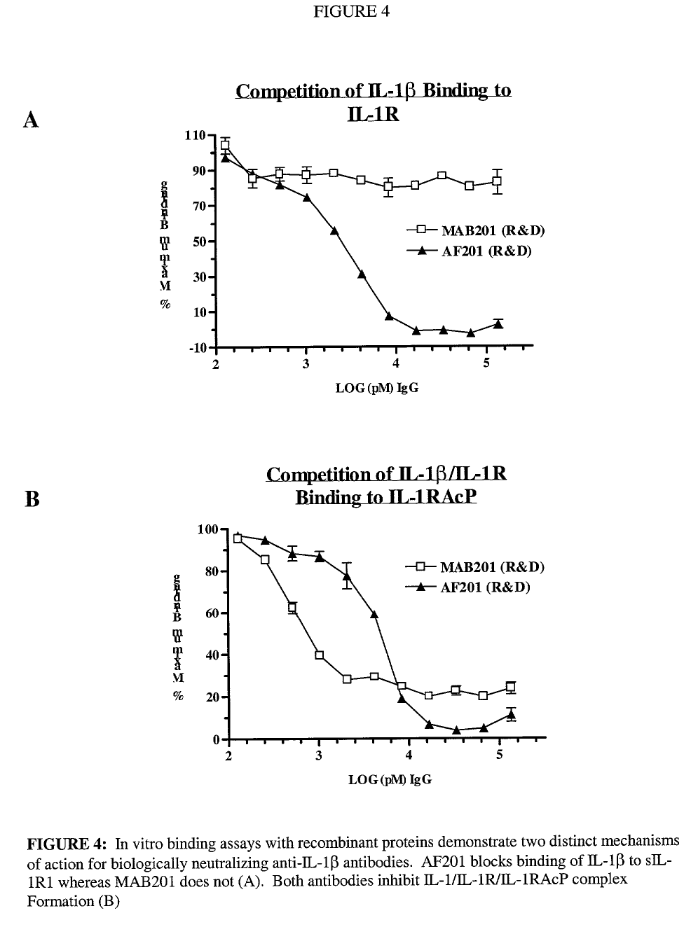

HUVEC and Synoviocyte assays were used to evaluate the relative potency of four distinct classes of inhibitors of IL-1 signaling pathway (FIGS. 2 to 3). The inhibition response curve for each inhibitor was established and IC50 was derived using Prism software. We found that anti-IL-1β antibodies are more potent than either IL-1ra, or examples of anti-IL-1R1 or anti-IL-RAcP that were tested. We also detected that one of the IL-1β antibodies was more potent than the other IL-1β antibody (AF201, which is a polyclonal antibody). Binding assays, using recombinant proteins to model the interactions between IL-1R1, IL-1β and IL-1RAcP, were used to determine the mechanism of action for each inhibitor (FIG. 4).

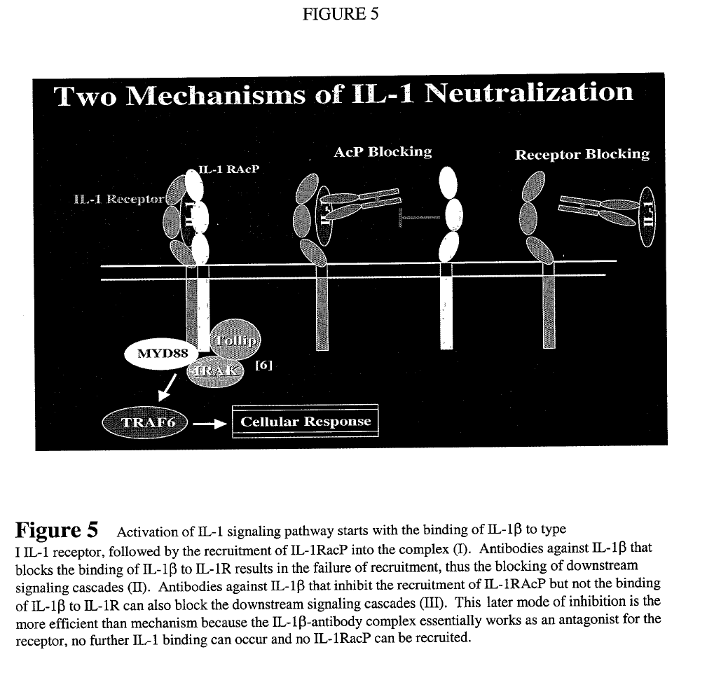

Based on these studies, we concluded that anti-IL-1β antibodies have two distinct mechanisms of action: (1) antibodies that inhibit IL-1 binding to IL-1R1, and (2) antibodies that allow IL-1R1 binding but block IL-1RAcP interaction. Antibodies against IL-1β that block binding of IL-1 to IL-1R1 result in the failure of heterodimerization of IL-1R1 with IL-1RAcP, thus blocking downstream signaling cascades (part II of FIG. 5). Antibodies against IL-1ν that allow binding of the bound IL-1β to IL-1R1 but inhibit recruitment of IL-1RAcP can also block the downstream signaling cascades (part III of FIG. 5). We concluded that the latter antibodies confer antagonist properties to the antibody-bound ligand (referred to herein as an antagonist complex).

This antagonist complex represents a novel method of IL-1 inhibition. This antagonist complex blocks signal transduction not only by the bound ligand but also by unbound ligand. The antagonist complex has IL-1ra-like properties—it blocks free IL-1β or IL-1α from binding to the receptor. In contrast, antibody that prevents antibody-bound IL-1β from binding to IL-1R1 does not prevent signal transduction by free IL-1β or IL-1α.

Definition of Terms

The term “selective binding agent” refers to a molecule which preferentially binds the ligand of interest (IL-1α or IL-1β herein). A selective binding agent may include a protein, peptide, nucleic acid, carbohydrate, lipid, or small molecular weight compound. In a preferred embodiment, a selective binding agent is an antibody, such as polyclonal antibodies, monoclonal antibodies (mAbs), chimeric antibodies, CDR-grafted antibodies, anti-idiotypic (anti-Id) antibodies to antibodies that can be labeled in soluble or bound form, as well as fragments, regions or derivatives thereof, provided by known techniques, including, but not limited to enzymatic cleavage, peptide synthesis or recombinant techniques. The IL-1 selective binding agents of the present invention are capable of binding IL-1α or β both and inhibiting signal transduction by unbound IL-1α and β.

The antibodies and antigen binding domains of the invention bind selectively to IL-1—i.e., they bind to IL-1 with a greater binding affinity than to other antigens. The antibodies may bind selectively to human IL-1, but also bind detectably to non-human IL-1, such as murine IL-1. Alternatively, the antibodies may bind selectively to non-human IL-1, but also bind detectably to human IL-1. Alternatively, the antibodies may bind exclusively to human IL-1, with no detectable binding to non-human IL-1.

The term “monoclonal antibody” refers to an antibody obtained from a population of substantially homogeneous antibodies wherein each monoclonal antibody will typically recognize a single epitope on the antigen. The term “monoclonal” is not limited to any particular method for making the antibody. For example, monoclonal antibodies of the invention may be made by the hybridoma method as described in Kohler et al. Nature 256, 495 (1975) or may be isolated from phage libraries using techniques as described herein, for example.

The terms “antigen binding domain” and “antigen binding region” refer to that portion of the selective binding agent (such as an antibody molecule) which contains the amino acid residues that interact with an antigen and confer on the binding agent its specificity and affinity for the antigen. Preferably, the antigen binding region will be of human origin.

In other embodiments, the antigen binding region can be derived from other animal species, in particular rodents such as rabbit, rat or hamster.

The term “epitope” refers to that portion of any molecule capable of being recognized by and bound by a selective binding agent (such as an antibody) at one or more of the binding agent's antigen binding regions. Epitopes usually consist of chemically active surface groupings of molecules, such as amino acids or sugar side chains, and have specific three-dimensional structural characteristics and specific charge characteristics. The term “inhibiting and/or neutralizing epitope” means an epitope that, when bound by a selective binding agent, results in loss of biological activity of the molecule or organism containing the epitope, in vivo, in vitro, or in situ, more preferably in vivo.

The term “light chain” when used in reference to an antibody refers to two distinct types, called kappa (κ) or lambda (λ) based on the amino acid sequence of the constant domains.

The term “heavy chain” when used in reference to an antibody refers to five distinct types, called alpha, delta, epsilon, gamma and mu, based on the amino acid sequence of the heavy chain constant domain. These distinct types of heavy chains give rise to five classes of antibodies, IgA, IgD, IgE, IgG and IgM, respectively, including four subclasses of IgG, namely IgG₁, IgG₂, IgG₃ and IgG₄.

The term “variable region” or “variable domain” refers to a portion of the light and heavy chains, typically about the amino-terminal 120 to 130 amino acids in the heavy chain and about the amino-terminal 100 to 110 amino acids in the light chain, which differ extensively in sequence among antibodies and are used in the binding and specificity of each particular antibody for its particular antigen. The variability in sequence is concentrated in those regions called complimentarily determining regions (CDRs) while the more highly conserved regions in the variable domain are called framework regions (FR). The CDRs of the light and heavy chains are responsible for the interaction of the antibody with antigen.

The term “constant region” or “constant domain” refers to a carboxy terminal portion of the light and heavy chain which is not directly involved in binding of the antibody to antigen but exhibits various effector function, such as interaction with the Fc receptor.

The term “IL-1” means that the text applies to IL-1α or IL-1β.

The term “IL-1α” or “IL-1α polypeptide” refers to a polypeptide comprising the amino acid sequence shown in SEQ ID NO: 1 below:

| | |
|---|---|
| 1 | MAKVPDMFED LKNCYSENEE DSSSIDHLSL NQKSFYHVSY GPLHEGCMDQ SVSLSISETS |
| 61 | KTSKLTFKES MVVVATNGKV LKKRRLSLSQ SITDDDLEAI ANDSEEEIIK PRSAPFSFLS |
| 121 | NVKYNFMRII KYEFILNDAL NQSIIRANDQ YLTAAALHNL DEAVKFDMGA YKSSKDDAKT |
| 181 | TVILRISKTQ LYVTAQDEDQ PVLLKEMPEI PKTITCSETN LLFFWETHGT KNYFTSVAHP |
| 241 | NLFIATKQDY WVCLAGGPPS ITDFQILENQ A |

IL-1α polypeptides are described in March et al. (1985), Nature 315:641-647; Furutani et al. (1986), Nucleic Acids Res. 14:3167-3179; Furutani et al. (1985), Nucleic Acids Res. 13:5869-5882; Kotenko et al. (1989), Dokl. Akad. Nauk SSSR 309:1005-1008; Gubler et al. (1986), J. Immunol. 136:2492-2497; Nishida et al. (1987), Biochem. Biophys. Res. Commun. 143:345-352; Zsebo et al. (1988), Blood 71:962-968; Graves et al. (1990), Biochemistry 29:2679-2684; Stevenson et al. (1993), Proc. Natl. Acad. Sci. U.S.A. 90:7245-7249. Each of these publications is hereby incorporated by reference. Related polypeptides include allelic variants; splice variants; fragments; derivatives; substitution, deletion, and insertion variants; fusion polypeptides; and interspecies homologs. IL-1α may be a mature polypeptide, as defined herein, and may or may not have an amino terminal methionine residue, depending upon the method by which it is prepared.

The term “IL-1β” or “IL-1β polypeptide” refers to a polypeptide comprising the amino acid sequence shown in SEQ ID NO: 2 below:

| | |
|---|---|
| 1 | MAEVPKLASE MMAYYSGNED DLFFEADGPK QMKCSFQDLD LCPLDGGIQL RISDEHYSKG |
| 61 | FRQAASVVVA MDRLRIMLVP CPQTFQENDL STFFPFIFEE EPIFFDTWDN EAYVHDAPVR |
| 121 | SLNCTLRDSQ QKSLVMSGPY ELKALHLQGQ DMEQQVVFSM SFVQGEESND KIPVALGLKE |
| 181 | KNLYLSCVLK DDKPTLQLES VDPKNYPKKK MEKRFVFNKI EINNKLEFES AQFPNWYIST |
| 241 | SQAENMPVFL GGTKGGQDIT DFTMQFVSS |

IL-1β polypeptides are described in Auron et al. (1984), Proc. Natl. Acad. Sci. U.S.A. 81:7907-7911; March et al. (1985), Nature 315:641-647; Clark et al. (1986), Nucleic Acids Res. 14:7897-7914; Bensi et al. (1987), Gene 52:95-101; Kotenko et al. (1989), Dokl. Akad. Nauk SSSR 309:1005-1008; Webb et al. (1985), Adv. Gene Technol. 22:339-340; Nishida et al. (1987), Biochem. Biophys. Res. Commun. 143:345-352; Zsebo et al. (1988), Blood 71:962-968; Priestle et al. (1988), EMBO J. 7:339-343; Priestle et al. (1989), Proc. Natl. Acad. Sci. U.S.A. 86:9667-9671; Finzel et al. (1989), J. Mol. Biol. 209:779-791; Vigers et al. (1997), Nature 386:190-194; Driscoll et al. (1990), Biochemistry 29:4668-4682; Clore et al. (1991), Biochemistry 30:2315-2323. Each of these publications is hereby incorporated by reference. Related polypeptides include allelic variants; splice variants; fragments; derivatives; substitution, deletion, and insertion variants; fusion polypeptides; and interspecies homologs. IL-1β may be a mature polypeptide, as defined herein, and may or may not have an amino terminal methionine residue, depending upon the method by which it is prepared.

The term “fragment” when used in relation to IL-1 or to a proteinaceous selective binding agent of IL-1 refers to a peptide or polypeptide that comprises less than the full length amino acid sequence. Such a fragment may arise, for example, from a truncation at the amino terminus, a truncation at the carboxy terminus, and/or an internal deletion of a residue(s) from the amino acid sequence. Fragments may result from alternative RNA splicing or from in vivo protease activity.

The term “variant” when used in relation to IL-1 or to a proteinaceous selective binding agent of IL-1 refers to a peptide or polypeptide comprising one or more amino acid sequence substitutions, deletions, and/or additions as compared to a native or unmodified sequence. For example, an IL-1 variant may result from one or more changes to an amino acid sequence of native IL-1. Also by way of example, a variant of a selective binding agent of IL-1 may result from one or more changes to an amino acid sequence of a native or previously unmodified selective binding agent. Variants may be naturally occurring, such as allelic or splice variants, or may be artificially constructed. Polypeptide variants may be prepared from the corresponding nucleic acid molecules encoding said variants.

The term “derivative” when used in relation to IL-1 or to a proteinaceous selective binding agent of IL-1 refers to a polypeptide or peptide, or a variant, fragment or derivative thereof, which has been chemically modified. Examples include covalent attachment of one or more polymers, such as water soluble polymers, N-linked, or O-linked carbohydrates, sugars, phosphates, and/or other such molecules. The derivatives are modified in a manner that is different from naturally occurring or starting peptide or polypeptides, either in the type or location of the molecules attached. Derivatives further include deletion of one or more chemical groups which are naturally present on the peptide or polypeptide.

The term “fusion” when used in relation to IL-1 or to a proteinaceous selective binding agent of IL-1 refers to the joining of a peptide or polypeptide, or fragment, variant and/or derivative thereof, with a heterologous peptide or polypeptide.

The term “biologically active” when used in relation to IL-1 or to a proteinaceous selective binding agent refers to a peptide or a polypeptide having at least one activity characteristic of IL-1 or a selective binding agent. A selective binding agent of IL-1 may have antagonist, or neutralizing or blocking activity with respect to at least one biological activity of IL-1. Such activity may be measured by assays described hereinafter or in WO 98/24477.

The term “naturally occurring” when used in connection with biological materials such as nucleic acid molecules, polypeptides, host cells, and the like, refers to those which are found in nature and not manipulated by a human being.

The term “isolated” when used in relation to IL-1 or to a proteinaceous selective binding agent of IL-1 refers to a peptide or polypeptide that is free from at least one contaminating polypeptide that is found in its natural environment, and preferably substantially free from any other contaminating mammalian polypeptides which would interfere with its therapeutic or diagnostic use.

The term “mature” when used in relation to IL-1 or to a proteinaceous selective binding agent of IL-1 refers to a peptide or polypeptide lacking a leader sequence. The term may also include other modifications of a peptide or polypeptide such as proteolytic processing of the amino terminus (with or without a leader sequence) and/or the carboxy terminus, cleavage of a smaller polypeptide from a larger precursor, N-linked and/or O-linked glycosylation, and the like.

The terms “effective amount” and “therapeutically effective amount” when used in relation to a selective binding agent of IL-1 refers to an amount of a selective binding agent that is useful or necessary to support an observable change in the level of one or more biological activities of IL-1. Said change may be either an increase or decrease in the level of IL-1 activity.

The term “conservative amino acid substitution” refers to a substitution of a native amino acid residue with a non-native residue such that there is little or no effect on the polarity or charge of the amino acid residue at that position. For example, a conservative substitution results from the replacement of a non-polar residue in a polypeptide with any other non-polar residue. Furthermore, any native residue in a polypeptide may also be substituted with alanine, as has been previously described for alanine scanning mutagenesis. Cunningham et al., Science 244: 1081-5 (1989). Exemplary rules for conservative amino acid substitutions are set forth in Table A.

TABLE A
Conservative Amino Acid Substitutions

| Original Residues | Exemplary Substitutions | Preferred Substitutions |
|---|---|---|
| Ala | Val, Leu, Ile | Val |
| Arg | Lys, Gln, Asn | Lys |
| Asn | Gln, His, Lys, Arg | Gln |
| Asp | Glu | Glu |
| Cys | Ser | Ser |
| Gln | Asn | Asn |
| Glu | Asp | Asp |
| Gly | Pro, Ala | Ala |
| His | Asn, Gln, Lys, Arg | Arg |
| Ile | Leu, Val, Met, Ala, | Leu |
| | Phe, Norleucine | |
| Leu | Norleucine, Ile, | Ile |
| | Val, Met, Ala, Phe | |
| Lys | Arg, Gln, Asn | Arg |
| Met | Leu, Phe, Ile | Leu |
| Phe | Leu, Val, Ile, Ala, | Leu |
| | Tyr | |
| Pro | Ala | Ala |
| Ser | Thr | Thr |
| Thr | Ser | Ser |
| Trp | Tyr, Phe | Tyr |
| Tyr | Trp, Phe, Thr, Ser | Phe |
| Val | Ile, Met, Leu, Phe, | Leu |
| | Ala, Norleucine | |

Conservative amino acid substitutions also encompass non-naturally occurring amino acid residues which are typically incorporated by chemical peptide synthesis rather than by synthesis in biological systems. These include peptidomimetics, and other reversed or inverted forms of amino acid moieties.

By making conservative modifications to the amino acid sequence or corresponding modifications to the encoding nucleotides, one can produce IL-1 polypeptides having functional and chemical characteristics similar to those of naturally occurring IL-1. Likewise, one can employ such conservative modifications to produce or proteinaceous selective IL-1 binding agents having functional and chemical characteristics similar to those of previously discovered selective binding agents. In contrast, substantial modifications in the functional and/or chemical characteristics of IL-1 (or of protineaceous IL-1-selective binding agents) may be accomplished by selecting substitutions that differ significantly in their effect on maintaining (a) the structure of the molecular backbone in the area of the substitution, for example, as a sheet or helical conformation, (b) the charge or hydrophobicity of the molecule at the target site, or (c) the bulk of the side chain. Naturally occurring residues may be divided into groups based on common side chain properties:

1) Hydrophobic: norleucine, Met, Ala, Val, Leu, Ile;

2) Neutral hydrophilic: Cys, Ser, Thr;

3) Acidic: Asp, Glu;

4) Basic: Asn, Gln, His, Lys, Arg;

5) Residues that influence chain orientation: Gly, Pro; and

6) Aromatic: Trp, Tyr, Phe.

Non-conservative substitutions may involve the exchange of a member of one of these classes for a member from another class.

The “identity or similarity” of two or more nucleic acid molecules and/or polypeptides provides a measure of the relatedness of two or more distinct sequences. The term “identity” refers to amino acids which are identical at corresponding positions in two distinct amino acid sequences. The term “similarity” refers to amino acids which are either identical or are conservative substitutions as defined above at corresponding positions in two distinct amino acid sequences.

The extent of identity or similarity can be readily calculated by known methods, including but not limited to those described in Computational Molecular Biology (Lesk, ed.), Oxford University Press, New York (1988); Biocomputing: Informatics and Genome Projects (Smith, ed.), Academic Press, New York, 1993; Computer Analysis of Sequence Data Part 1 (Griffin, A. M., and Griffin, H. G., eds.), Humana Press, New Jersey (1994); von Heinje, Sequence Analysis in Molecular Biology, Academic Press (1987); Sequence Analysis Primer (Gribskov and Devereux, eds.), M. Stockton Press, New York (1991); and Carillo et al., SIAM J. Applied Math., 48: 1073 (1988).

Preferred methods to determine identity and/or similarity are designed to give the largest match between the sequences tested. Methods to determine identity and similarity are codified in publicly available computer programs. Exemplary computer program methods to determine identity and similarity between two sequences include, but are not limited to, the GCG program package, including GAP (Devereux et al., Nucleic Acids Research 12: 387 (1984)); BLASTP, BLASTN, and FASTA (Altschul et al., J. Mol. Biol., 215: 403-10 (1990)). The BLAST X program is publicly available from the National Center for Biotechnology Information (NCBI) and other sources (BLAST Manual, Altschul et al. NCB NLM NIH Bethesda, Md.). The well known Smith-Waterman algorithm may also be used to determine identity.

IL-1 polypeptides

IL-1 polypeptides and fragments, variants and derivatives thereof, are used as target molecules for screening and identifying the selective binding agents of the invention. When the selective binding agents are antibodies, IL-1 polypeptides are preferably immunogenic—that is, they elicit an immune response when administered to an animal. Alternatively, when antibodies are prepared by in vitro techniques, IL-1 polypeptides used as target molecules are capable of detectably binding an antibody or antigen binding domain.

IL-1 polypeptides are prepared by biological or chemical methods. Biological methods such as expression of DNA sequences encoding recombinant IL-1 are known in the art (see for example Sambrook et al. supra). Chemical synthesis methods may also be used to prepare IL-1 polypeptides of the invention. See, for example, the methods set forth in Merrifield et al. (1963), J. Am. Chem. Soc., 85: 2149, Houghten et al. (1985), Proc Natl Acad. Sci. USA, 82: 5132, and Stewart and Young (1984), Solid Phase Peptide Synthesis, Pierce Chemical Co., Rockford, Ill. Such polypeptides may be synthesized with or without a methionine on the amino terminus. Chemically synthesized IL-1 polypeptides, or fragments or variants thereof, may be oxidized using methods set forth in these references to form disulfide bridges. IL-1 polypeptides of the invention prepared by chemical synthesis will have at least one biological activity comparable to the corresponding IL-1 polypeptides produced recombinantly or purified from natural sources.

IL-1 polypeptides may be obtained by isolation from biological samples such as source tissues and/or fluids in which the IL-1 polypeptides are naturally found. Sources for IL-1 polypeptides may be human or non-human in origin. Isolation of naturally-occurring IL-1 polypeptides can be accomplished using methods known in the art, such as separation by electrophoresis followed by electroelution, various types of chromatography (affinity, immunoaffinity, molecular sieve, and/or ion exchange), and/or high pressure liquid chromatography. The presence of the IL-1 polypeptide during purification may be monitored using, for example, an antibody prepared against recombinantly produced IL-1 polypeptide or peptide fragments thereof.

Polypeptides of the invention include isolated IL-1 polypeptides and polypeptides related thereto including fragments, variants, fusion polypeptides, and derivatives as defined hereinabove. IL-1 fragments of the invention may result from truncations at the amino terminus (with or without a leader sequence), truncations at the carboxy terminus, and/or deletions internal to the polypeptide. Such IL-1 polypeptides fragments may optionally comprise an amino terminal methionine residue. The polypeptides of the invention will be immunogenic in that they will be capable of eliciting an antibody response.

IL-1 polypeptide variants of the invention include one or more amino acid substitutions, additions and/or deletions as compared to the native IL-1 amino acid sequence. Amino acid substitutions may be conservative, as defined above, or non-conservative or any combination thereof. The variants may have additions of amino acid residues either at the carboxy terminus or at the amino terminus (where the amino terminus may or may not comprise a leader sequence).

Embodiments of the invention include IL-1 glycosylation variants and cysteine variants. IL-1 glycosylation variants include variants wherein the number and/or type of glycosylation sites has been altered compared to native IL-1 polypeptide. In one embodiment, IL-1 glycosylation variants comprise a greater or a lesser number of N-linked glycosylation sites compared to native IL-1. Also provided for are IL-1 glycoyslation variants comprising a rearrangement of N-linked carbohydrate chains wherein one or more N-linked glycosylation sites (typically those that are naturally occurring) are eliminated and one or more new N-linked sites are created. IL-1 cysteine variants comprise a greater number or alternatively a lesser number of cysteine residues compared to native IL-1. In one embodiment, one or more cysteine residues are deleted or substituted with another amino acid (e.g., serine). Cysteine variants of IL-1 can improve the recovery of biologically active IL-1 by aiding the refolding of IL-1 into a biologically active conformation after isolation from a denatured state.

Preparing IL-1 polypeptide variants is within the level of skill in the art. In one approach, one may introduce one or more amino acid substitutions, deletions and/or additions in native IL-1 wherein the IL-1 variant retains the native structure of IL-1 and/or at least one of the biological activities. One approach is to compare sequences of IL-1 polypeptides from a variety of different species in order to identify regions of relatively low and high identity and/or similarity. It is appreciated that those regions of an IL-1 polypeptide having relatively low identity and/or similarity, are less likely to be essential for structure and activity and therefore may be more tolerant of amino acid alterations, especially those which are non-conservative. It is also appreciated that even in relatively conserved regions, one could introduce conservative amino acid substitutions while retaining activity.

In another approach, structure-function relationships can be used to identify residues in similar polypeptides that are important for activity or structure. For example, one may compare conserved amino acid residues among IL-1 and other members of the IL-1 family for which structure-function analyses are available and, based on such a comparison, predict which amino acid residues in IL-1 are important for activity or structure. One skilled in the art may choose chemically similar amino acid substitutions for such predicted important amino acid residues of IL-1.

In yet another approach, an analysis of a secondary or tertiary structure of IL-1 (either determined by x-ray diffraction of IL-1 crystals or by structure prediction methods) can be undertaken to determine the location of specific amino acid residues in relation to actual or predicted structures within an IL-1 polypeptide. Using this information, one can introduce amino acid changes in a manner that seeks to retain as much as possible the secondary and/or tertiary structure of an IL-1 polypeptide.

In yet another approach, the effects of altering amino acids at specific positions may be tested experimentally by introducing amino acid substitutions and testing the altered IL-1 polypeptides for biological activity using assays described herein. Techniques such as alanine scanning mutagenesis (Cunningham et al., supra) are particularly suited for this approach. Many altered sequence may be conveniently tested by introducing many substitutions at various amino acid positions in IL-1 and screening the population of altered polypeptides as part of a phage display library. Using this approach, those regions of an IL-1 polypeptide that are essential for activity may be readily determined.

The above methods are useful for generating IL-1 variants which retain the native structure. Thus, antibodies raised against each variants are likely to recognize a native structural determinant, or epitope, of IL-1 and are also likely to bind to native IL-1. However, in some cases is may be desirable to produce IL-1 variants which do not retain native IL-1 structure or are partially or completely unfilled. Antibodies raised against such proteins will recognize buried epitopes on IL-1.

The invention also provides for IL-1 fusion polypeptides which comprise IL-1 polypeptides, and fragments, variants, and derivatives thereof, fused to a heterologous peptide or protein. Heterologous peptides and proteins include, but are not limited to: an epitope to allow for detection and/or isolation of a IL-1 fusion polypeptide; a transmembrane receptor protein or a portion thereof, such as an extracellular domain, or a transmembrane and intracellular domain; a ligand or a portion thereof which binds to a transmembrane receptor protein; an enzyme or portion thereof which is catalytically active; a protein or peptide which promotes oligomerization, such as leucine zipper domain; and a protein or peptide which increases stability, such as an immunoglobulin constant region. An IL-1 polypeptide may be fused to itself or to a fragment, variant, or derivative thereof. Fusions may be made either at the amino terminus or at the carboxy terminus of an IL-1 polypeptide, and may be direct with no linker or adapter molecule or may be through a linker or adapter molecule. A linker or adapter molecule may also be designed with a cleavage site for a DNA restriction endonuclease or for a protease to allow for separation of the fused moieties.

IL-1 Selective Binding Agents

IL-1 polypeptides, and fragments, variants and derivatives thereof, may be used to identify selective binding agents of IL-1. As defined above, a selective binding agent of IL-1 encompasses both proteinaceous and non-proteinaceous binding agents and, in one preferred embodiment of the invention, the selective binding agent is proteinaceous. In yet another preferred embodiment, the selective binding agent is an antibody or fragment thereof which binds IL-1, preferably human IL-1, more preferably human IL-1β. The antagonist antibodies of the present invention may also be referred to as inhibitory or neutralizing antibodies of IL-1. Although such antibodies are preferred embodiments of the invention, it is understood that other proteinaceous selective binding agents are also encompassed by the invention.

As described in the examples below, anti-IL-1 antibodies and antigen binding domains which inhibit at least one activity of IL-1 have been identified. The antibodies of the invention comprise a human Fc region from any isotype, either IgG, IgM, IgA, IgE, or IgD. Preferably, the Fc region is from human IgG, such as IgG1, IgG2, IgG3, or IgG4.

The invention also provides for antibodies or antigen binding domains which comprise fragments, variants, or derivatives of the Fab sequences disclosed herein. Fragments include variable domains of either the light or heavy chain Fab sequences which are typically joined to light or heavy constant domains. Variants include antibodies comprising one or more of the following:

light chain Fab sequences that are at least about 80%, 85%, 90%, 95%, 98% or 99% identical or similar to the Fab sequences, or the corresponding variable domains, of any of the antibodies exemplified herein; or

heavy chain Fab sequences, or the corresponding variable domains, that are at least about 80%, 85%, 90%, 95%, 98% or 99% identical or similar to the Fab sequences in any one of of any of the antibodies exemplified herein.

The antibodies may be typically associated with constant regions of the heavy and light chains to form full-length antibodies.

Antibodies and antigen binding domains, and fragments, variants and derivatives thereof, of the invention will retain the ability to bind selectively to an IL-1 polypeptide, preferably to a human IL-1 polypeptide.

In one embodiment, an antibody will bind an IL-1 polypeptide with a dissociation constant (KD) of about 1 nM or less, or alternatively 0.1 nM or less, or alternatively 10 pM or less or alternatively less than 10 pM.

Antibodies of the invention include polyclonal monospecific polyclonal, monoclonal, recombinant, chimeric, humanized, fully human, single chain and/or bispecific antibodies. Antibody fragments include those portions of an anti-IL-1 antibody which bind to an epitope on an IL-1 polypeptide. Examples of such fragments include Fab F(ab′), F(ab)′, Fv, and sFv fragments. The antibodies may be generated by enzymatic cleavage of full-length antibodies or by recombinant DNA techniques, such as expression of recombinant plasmids containing nucleic acid sequences encoding antibody variable regions.

Polyclonal antibodies are heterogeneous populations of antibody molecules derived from the sera of animals immunized with an antigen. An antigen is a molecule or a portion of a molecule capable of being bound by an antibody which is additionally capable of inducing an animal to produce antibody capable of binding to an epitope of that antigen. An antigen can have one or more epitope. The specific reaction referred to above is meant to indicate that the antigen will react, in a highly selective manner, with its corresponding antibody and not with the multitude of other antibodies which can be evoked by other antigens.

Polyclonal antibodies directed toward an IL-1 polypeptide generally are raised in animals (e.g., rabbits or mice) by multiple subcutaneous or intraperitoneal injections of IL-1 and an adjuvant. In accordance with the invention, it may be useful to conjugate an IL-1 polypeptide, or a variant, fragment, or derivative thereof to a carrier protein that is immunogenic in the species to be immunized, such as keyhole limpet heocyanin, serum, albumin, bovine thyroglobulin, or soybean trypsin inhibitor. Also, aggregating agents such as alum are used to enhance the immune response. After immunization, the animals are bled and the serum is assayed for anti-IL-1 antibody titer.

Monoclonal antibodies (mAbs) contain a substantially homogeneous population of antibodies specific to antigens, which population contains substantially similar epitope binding sites. Such antibodies may be of any immunoglobulin class including IgG, IgM, IgE, IgA, IgD and any subclass thereof. A hybridoma producing a monoclonal antibody of the present invention may be cultivated in vitro, in situ, or in vivo. Production of high titers in vivo or in situ is a preferred method of production.

Monoclonal antibodies directed toward IL-1 are produced using any method which provides for the production of antibody molecules by continuous cell lines in culture. Examples of suitable methods for preparing monoclonal antibodies include hybridoma methods of Kohler et al. (1975), Nature 256: 495-7, and the human B-cell hybridoma method, Kozbor (1984), J. Immunol. 133: 3001; Brodeur et al. (1987), Monoclonal Antibody Production Techniques and Applications, Marcel Dekker, Inc., New York, pp. 51-63; and Harlow and Lane (1988), Antibodies: A Laboratory Manual, Cold Spring Harbor Laboratory. The contents of these references are incorporated entirely herein by reference.

Preferred methods for determining monoclonal antibody specificity and affinity by competitive inhibition can be found in Harlow and Lane (1988); Current Protocols in Immunology (1992, 1993; Colligan et al., eds.), Greene Publishing Assoc. and Wiley Interscience, N.Y.; and Muller (1983), Meth. Enzymol., 92: 589-601. These references are incorporated herein by reference.

Also provided by the invention are hybridoma cell lines which produce monoclonal antibodies reactive with IL-1 polypeptides.

Chimeric antibodies are molecules in which different portions are derived from different animal species, such as those having a variable region derived from a murine monoclonal antibody and a human immunoglobulin constant region. Chimeric antibodies are primarily used to reduce immunogenicity in application and to increase yields in production, for example, where murine monoclonal antibodies have higher yields from hybridomas but higher immunogenicity in humans, such that human/murine chimeric monoclonal antibodies are used.

Chimeric antibodies and methods for their production are known in the art. Cabilly et al. (1984), Proc. Natl. Acad. Sci. USA, 81: 3273-7; Morrison et al. (1984), Proc. Natl. Acad. Sci. USA, 81: 6851-5; Boulianne et al. (1984), Nature, 312: 643-6; Neuberger et al. (1985), Nature, 314: 268-70; Liu et al. (1987), Proc. Natl. Acad. Sci. USA, 84: 3439-43; and Harlow and Lane (1988), Antibodies: A Laboratory Manual, Cold Spring Harbor Laboratory. These references are incorporated herein by reference.

For example, chimeric monoclonal antibodies of the invention may be used as a therapeutic. In such a chimeric antibody, a portion of the heavy and/or light chain is identical with or homologous to corresponding sequence in antibodies derived from a particular species or belonging to one particular antibody class or subclass, while the remainder of the chain(s) is identical with or homologous to corresponding sequence in antibodies derived from another species or belonging to another antibody class or subclass, as well as fragments of such antibodies, so long as they exhibit the desired biological activity (see U.S. Pat. No. 4,816,567; Morrison et al. (1985), Proc. Natl. Acad. Sci., 81: 6851-5.

As used herein, the term “chimeric antibody” includes monovalent, divalent or polyvalent immunoglobulins. A monovalent chimeric antibody is a dimer (HL) formed by a chimeric H chain associated through disulfide bridges with a chimeric L chain. A divalent chimeric antibody is tetramer (H₂L₂) formed by two HL dimers associated through at least one disulfide bridge. A polyvalent chimeric antibody can also be produced, for example, by employing a C_{H} region that aggregates (e.g., from an IgM H chain, or μ chain).

Murine and chimeric antibodies, fragments and regions of the present invention may comprise individual heavy (H) and/or light (L) immunoglobulin chains. A chimeric H chain comprises an antigen binding region derived from the H chain of a non-human antibody specific for IL-1, which is linked to at least a portion of a human H chain C region (C_{H}), such as CH₁ or CH₂.

A chimeric L chain according to the present invention comprises an antigen binding region derived from the L chain of a non-human antibody specific for IL-1, linked to at least a portion of a human L chain C region (C_{L}).

Selective binding agents, such as antibodies, fragments, or derivatives, having chimeric H chains and L chains of the same or different variable region binding specificity, can also be prepared by appropriate association of the individual polypeptide chains, according to known method steps, e.g., according to Current Protocols in Molecular Biology (1993) and Harlow et al. (1988). The contents of these references are incorporated entirely herein by reference. With this approach, hosts expressing chimeric H chains (or their derivatives) are separately cultured from hosts expressing chimeric L chains (or their derivatives), and the immunoglobulin chains are separately recovered and then associated. Alternatively, the hosts can be co-cultured and the chains allowed to associate spontaneously in the culture medium, followed by recovery of the assembled immunoglobulin, fragment or derivative.

As an example, the antigen binding region of the selective binding agent (such as a chimeric antibody) of the present invention is preferably derived from a non-human antibody specific for human IL-1. Preferred sources for the DNA encoding such a non-human antibody include cell lines which produce antibodies, such as hybrid cell lines commonly known as hybridomas.

The invention also provides for fragments, variants and derivatives, and fusions of anti-IL-1 antibodies, wherein the terms “fragments”, “variants”, “derivatives” and “fusions” are defined herein. The invention encompasses fragments, variants, derivatives, and fusions of anti-IL-1 antibodies which are functionally similar to the unmodified anti-IL-1 antibody, that is, they retain at least one of the activities of the unmodified antibody. In addition to the modifications set forth above, also included is the addition of genetic sequences coding for cytotoxic proteins such as plant and bacterial toxins. The fragments, variants, derivatives and fusions of anti-IL-1 antibodies can be produced from any of the hosts of this invention.

Suitable fragments include, for example, Fab, Fab′, F(ab′)₂, Fv and scFv. These fragments lack the Fc fragment of an intact antibody, clear more rapidly from the circulation, and can have less non-specific tissue binding than an intact antibody. See Wahl et al. (1983), J. Nucl. Med., 24: 316-25. These fragments are produced from intact antibodies using methods well known in the art, for example by proteolytic cleavage with enzymes such as papain (to produce Fab fragments) or pepsin (to produce F(ab′)₂ fragments). The identification of these antigen binding regions and/or epitopes recognized by monoclonal antibodies of the present invention provides the information necessary to generate additional monoclonal antibodies with similar binding characteristics and therapeutic or diagnostic utility that parallel the embodiments of this application.

Variants of selective binding agents are also provided. In one embodiment, variants of antibodies and antigen binding domains comprise changes in light and/or heavy chain amino acid sequences that are naturally occurring or are introduced by in vitro engineering of native sequences using recombinant DNA techniques. Naturally occurring variants include “somatic” variants which are generated in vivo in the corresponding germ line nucleotide sequences during the generation of an antibody response to a foreign antigen.

Variants of anti-IL-1 antibodies and antigen binding domains are also prepared by mutagenesis techniques known in the art. In one example, amino acid changes may be introduced at random throughout an antibody coding region and the resulting variants may be screened for a desired activity, such as binding affinity for IL-1. Alternatively, amino acid changes may be introduced in selected regions of an IL-1 antibody, such as in the light and/or heavy chain CDRs, and framework regions, and the resulting antibodies may be screened for binding to IL-1 or some other activity. Amino acid changes encompass one or more amino acid substitutions in a CDR, ranging from a single amino acid difference to the introduction of all possible permutations of amino acids within a given CDR, such as CDR3. In another method, the contribution of each residue within a CDR to IL-1 binding may be assessed by substituting at least one residue within the CDR with alanine. Lewis et al. (1995), Mol. Immunol. 32: 1065-72. Residues which are not optimal for binding to IL-1 may then be changed in order to determine a more optimum sequence. Also encompassed are variants generated by insertion of amino acids to increase the size of a CDR, such as CDR3. For example, most light chain CDR3 sequences are nine amino acids in length. Light chain CDR3 sequences in an antibody which are shorter than nine residues may be optimized for binding to IL-1 by insertion of appropriate amino acids to increase the length of the CDR.

In one embodiment, antibody or antigen binding domain variants comprise one or more amino acid changes in one or more of the heavy or light chain CDR1, CDR2 or CDR3 and optionally one or more of the heavy or light chain framework regions FR1, FR2 or FR3. Amino acid changes comprise substitutions, deletions and/or insertions of amino acid residues. Exemplary variants include an “AT” heavy chain variable region variant or an “AT” light chain variable region variant. The “AT” heavy and light chain variable region variants may further comprise one or more amino acid changes in the framework regions. In one example, one or more amino acid changes may be introduced to substitute a somatically mutated framework residue with the germline residue at that position. When the aforementioned amino acid changes are substitutions, the changes may be conservative or non-conservative substitutions.

Variants may also be prepared by “chain shuffling” of either light or heavy chains. Marks et al. (1992), Biotechnology 10: 779-83. Typically, a single light (or heavy) chain is combined with a library having a repertoire of heavy (or light) chains and the resulting population is screened for a desired activity, such as binding to IL-1. This technique permits screening of a greater sample of different heavy (or light) chains in combination with a single light (or heavy) chain than is possible with libraries comprising repertoires of both heavy and light chains.

The selective binding agents of the invention can be bispecific. Bispecific selective binding agents of this invention can be of several configurations. For example, bispecific antibodies resemble single antibodies (or antibody fragments) but have two different antigen binding sites (variable regions). Bispecific antibodies can be produced by chemical techniques (see e.g., Kranz et al. (1981), Proc. Natl. Acad. Sci. USA, 78: 5807), by “polydoma” techniques (see U.S. Pat. No. 4,474,893 to Reading) or by recombinant DNA techniques.

The selective binding agents of the invention may also be heteroantibodies. Heteroantibodies are two or more antibodies, or antibody binding fragments (Fab) linked together, each antibody or fragment having a different specificity.

The invention also relates to “humanized” antibodies. Methods for humanizing non-human antibodies are well known in the art. Generally, a humanized antibody has one or more amino acid residues introduced into a human antibody from a source which is non-human. In general, non-human residues will be present in CDRs. Humanization can be performed following methods known in the art. Jones et al. (1986), Nature 321: 522-5; Riechmann et al. (1988), Nature, 332: 323-7; Verhoeyen et al. (1988), Science 239: 1534-6. One may carry out such methods by substituting rodent CDRs for the corresponding regions of a human antibody.

The selective binding agents of the invention, including chimeric, CDR-grafted, fully human, and humanized antibodies can be produced by recombinant methods known in the art. Nucleic acids encoding the antibodies are introduced into host cells and expressed using materials and procedures described herein and known in the art. In a preferred embodiment, the antibodies are produced in mammalian host cells, such as CHO cells. Fully human antibodies may be produced by expression of recombinant DNA transfected into host cells or by expression in hybridoma cells as described above.

Techniques for creating recombinant DNA versions of the antigen-binding regions of antibody molecules which bypass the generation of monoclonal antibodies are encompassed within the practice of this invention. To do so, antibody-specific messenger RNA molecules are extracted from immune system cells taken from an immunized animal, and transcribed into complementary DNA (cDNA). The cDNA is then cloned into a bacterial expression system. One example of such a technique suitable for the practice of this invention uses a bacteriophage lambda vector system having a leader sequence that causes the expressed Fab protein to migrate to the periplasmic space (between the bacterial cell membrane and the cell wall) or to be secreted. One can rapidly generate and screen great numbers of functional Fab fragments for those which bind the antigen. Such IL-1 selective binding agents (Fab fragments with specificity for an IL-1 polypeptide) are specifically encompassed within the term “antibody” as it is defined, discussed, and claimed herein.

Also within the scope of the invention are techniques developed for the production of chimeric antibodies by splicing the genes from a mouse antibody molecule of appropriate antigen-specificity together with genes from a human antibody molecule of appropriate biological activity, such as the ability to activate human complement and mediate ADCC. Morrison et al. (1984), Proc. Natl. Acad. Sci., 81: 6851; Neuberger et al. (1984), Nature, 312: 604. One example is the replacement of a Fc region with that of a different isotype. Selective binding agents such as antibodies produced by this technique are within the scope of the invention.

In a preferred embodiment of the invention, the anti-IL-1 antibodies are fully human antibodies. Thus encompassed by the invention are antibodies which bind IL-1 polypeptides and are encoded by nucleic acid sequences which are naturally occurring somatic variants of human germline immunoglobulin nucleic acid sequence, and fragments, synthetic variants, derivatives and fusions thereof. Such antibodies may be produced by any method known in the art. Exemplary methods include immunization with a IL-1 antigen (any IL-1 polypeptide capable of elicing an immune response, and optionally conjugated to a carrier) of transgenic animals (e.g., mice) that are capable of producing a repertoire of human antibodies in the absence of endogenous immunoglobulin production. See, for example, Jakobovits et al. (1993), Proc. Natl. Acad. Sci., 90: 2551-5; Jakobovits et al. (1993), Nature, 362: 255-8; Bruggermann et al. (1993), Year in Immunol., 7: 33.

Alternatively, human antibodies may be generated through the in vitro screening of phage display antibody libraries. See Hoogenboom et al. (1991), J. Mol. Biol. 227: 381; and Marks et al. (1991), J. Mol. Biol. 222: 581, which are incorporated herein by reference. Various antibody-containing phage display libraries have been described and may be readily prepared by one skilled in the art. Libraries may contain a diversity of human antibody sequences, such as human Fab, Fv, and scFv fragments, that may be screened against an appropriate target. Example 1 describes the screening of a Fab phage library against IL-1 to identify those molecules which selectively bind IL-1. It will be appreciated that phage display libraries may comprise peptides or proteins other than antibodies which may be screened to identify selective binding agents of IL-1.

An anti-idiotypic (anti-Id) antibody is an antibody which recognizes unique determinants generally associated with the antigen-binding site of an antibody. An Id antibody can be prepared by immunizing an animal of the same species and genetic type (e.g., mouse strain) as the source of the monoclonal antibody with the monoclonal antibody to which an anti-Id is being prepared. The immunized animal will recognize and respond to the idiotypic determinants of the immunizing antibody by producing an antibody to these idiotypic determinants (the anti-Id antibody). See, for example, U.S. Pat. No. 4,699,880, which is herein incorporated by reference in its entirety. The anti-Id antibody may also be used as an “immunogen” to induce an immune response in yet another animal, producing a so-called anti-anti-Id antibody. The anti-anti-Id may be epitopically identical to the original monoclonal antibody which induced the anti-Id. Thus, by using antibodies to the idiotypic determinants of a mAb, it is possible to identify other clones expressing antibodies of identical specificity.

The selective binding agents may also be peptides or peptide fusion proteins. Peptides may be generated by phage display and related techniques as described in WO 99/25044. Such specific binding peptides may be used directly or incorporated in fusion proteins with, for example, an immunoglobulin constant region (Fc domain) or human serum albumin (HAS). Techniques for fusion with Fc are reviewed in WO 99/25044 (previously cited and incorporated by reference). Fusion with HSA is described in U.S. Pat. No. 5,876,969, which is hereby incorporated by reference.

Production of Selective Binding Agents of IL-1

When the selective binding agent of IL-1 to be prepared is a proteinaceous selective binding agent, such as an antibody or an antigen binding domain, various biological or chemical methods for producing said agent are available.

Biological methods are preferable for producing sufficient quantities of a selective binding agent for therapeutic use. Standard recombinant DNA techniques are particularly useful for the production of antibodies and antigen binding domains of the invention. Exemplary expression vectors, host cells and methods for recovery of the expressed product are described below.

A nucleic acid molecule encoding an IL-1 antibody or antigen binding domain is inserted into an appropriate expression vector using standard ligation techniques. The vector is typically selected to be functional in the particular host cell employed (i.e., the vector is compatible with the host cell machinery such that amplification of the gene and/or expression of the gene can occur). A nucleic acid molecule encoding an anti-IL-1 antibody may be amplified/expressed in prokaryotic, yeast, insect (baculovirus systems) and/or eukaryotic host cells. Selection of the host cell will depend in part on whether an anti-IL-1 antibody is to be post-transitionally modified (e.g., glycosylated and/or phosphorylated). If so, yeast, insect, or mammalian host cells are preferable. For a review of expression vectors, see Meth. Enz. v. 185 (1990; Goeddel, ed.), Academic Press Inc., San Diego, Calif.

Typically, expression vectors used in any host cells will contain one or more of the following components: a promoter, one or more enhancer sequences, an origin of replication, a transcriptional termination sequence, a complete intron sequence containing a donor and acceptor splice site, a leader sequence for secretion, a ribosome binding site, a polyadenylation sequence, a polylinker region for inserting the nucleic acid encoding the polypeptide to be expressed, and a selectable marker element. Each of these sequences is discussed in more detail below.

The vector components may be homologous (i.e., from the same species and/or strain as the host cell), heterologous (i.e., from a species other than the host cell species or strain), hybrid (i.e., a combination of different sequences from more than one source), synthetic, or native sequences which normally function to regulate immunoglobulin expression. As such, a source of vector components may be any prokaryotic or eukaryotic organism, any vertebrate or invertebrate organism, or any plant, provided that the components are functional in, and can be activated by, the host cell machinery.

An origin of replication is selected based upon the type of host cell being used for expression. For example, the origin of replication from the plasmid pBR322 (Product No. 303-3s, New England Biolabs, Beverly, Mass.) is suitable for most Gram-negative bacteria while various origins from SV40, polyoma, adenovirus, vesicular stomatitus virus (VSV) or papillomaviruses (such as HPV or BPV) are useful for cloning vectors in mammalian cells. Generally, the origin of replication component is not needed for mammalian expression vectors (for example, the SV40 origin is often used only because it contains the early promoter).

A transcription termination sequence is typically located 3′ of the end of a polypeptide coding regions and serves to terminate transcription. Usually, a transcription termination sequence in prokaryotic cells is a G-C rich fragment followed by a poly T sequence. While the sequence is easily cloned from a library or even purchased commercially as part of a vector, it can also be readily synthesized using methods for nucleic acid synthesis such as those described above.

A selectable marker gene element encodes a protein necessary for the survival and growth of a host cell grown in a selective culture medium. Typical selection marker genes encode proteins that (a) confer resistance to antibiotics or other toxins, e.g., ampicillin, tetracycline, or kanamycin for prokaryotic host cells, (b) complement auxotrophic deficiencies of the cell; or (c) supply critical nutrients not available from complex media. Preferred selectable markers are the kanamycin resistance gene, the ampicillin resistance gene, and the tetracycline resistance gene. A neomycin resistance gene may also be used for selection in prokaryotic and eukaryotic host cells.

Other selection genes may be used to amplify the gene which will be expressed. Amplification is the process wherein genes which are in greater demand for the production of a protein critical for growth are reiterated in tandem within the chromosomes of successive generations of recombinant cells. Examples of suitable selectable markers for mammalian cells include dihydrofolate reductase (DHFR) and thymidine kinase. The mammalian cell transformants are placed under selection pressure which only the transformants are uniquely adapted to survive by virtue of the marker present in the vector. Selection pressure is imposed by culturing the transformed cells under conditions in which the concentration of selection agent in the medium is successively changed, thereby leading to amplification of both the selection gene and the DNA that encodes an anti-IL-1 antibody. As a result, increased quantities of an antibody are synthesized from the amplified DNA.

A ribosome binding site is usually necessary for translation initiation of mRNA and is characterized by a Shine-Dalgarno sequence (prokaryotes) or a Kozak sequence (eukaryotes). The element is typically located 3′ to the promoter and 5′ to the coding sequence of the polypeptide to be expressed. The Shine-Dalgarno sequence is varied but is typically a polypurine (i.e., having a high A-G content). Many Shine-Dalgarno sequences have been identified, each of which can be readily synthesized using methods set forth above and used in a prokaryotic vector.

A leader, or signal, sequence is used to direct secretion of a polypeptide. A signal sequence may be positioned within or directly at the 5′ end of a polypeptide coding region. Many signal sequences have been identified and may be selected based upon the host cell used for expression. In the present invention, a signal sequence may be homologous (naturally occurring) or heterologous to a nucleic acid sequence encoding an anti-IL-1 antibody or antigen binding domain. A heterologous signal sequence selected should be one that is recognized and processed, i.e., cleaved, by a signal peptidase, by the host cell. For prokaryotic host cells that do not recognize and process a native immunoglobulin signal sequence, the signal sequence is substituted by a prokaryotic signal sequence selected, for example, from the group of the alkaline phosphatase, penicillinase, or heat-stable enterotoxin II leaders. For yeast secretion, a native immunoglobulin signal sequence may be substituted by the yeast invertase, alpha factor, or acid phosphatase leaders. In mammalian cell expression the native signal sequence is satisfactory, although other mammalian signal sequences may be suitable.

In most cases, secretion of an anti-IL-1 antibody or antigen binding domain from a host cell will result in the removal of the signal peptide from the antibody. Thus the mature antibody will lack any leader or signal sequence.

In some cases, such as where glycosylation is desired in a eukaryotic host cell expression system, one may manipulate the various presequences to improve glycosylation or yield. For example, one may alter the peptidase cleavage site of a particular signal peptide, or add prosequences, which also may affect glycosylation. The final protein product may have, in the −1 position (relative to the first amino acid of the mature protein) one or more additional amino acids incident to expression, which may not have been totally removed. For example, the final protein product may have one or two amino acid found in the peptidase cleavage site, attached to the N-terminus. Alternatively, use of some enzyme cleavage sites may result in a slightly truncated form of the desired IL-1 polypeptide, if the enzyme cuts at such area within the mature polypeptide.

The expression vectors of the present invention will typically contain a promoter that is recognized by the host organism and operably linked to a nucleic acid molecule encoding an anti-IL-1 antibody or antigen binding domain. Either a native or heterologous promoter may be used depending the host cell used for expression and the yield of protein desired.

Promoters suitable for use with prokaryotic hosts include the beta-lactamase and lactose promoter systems; alkaline phosphatase, a tryptophan (trp) promoter system; and hybrid promoters such as the tac promoter. Other known bacterial promoters are also suitable. Their sequences have been published, thereby enabling one skilled in the art to ligate them to the desired DNA sequence(s), using linkers or adapters as needed to supply any required restriction sites.

Suitable promoters for use with yeast hosts are also well known in the art. Yeast enhancers are advantageously used with yeast promoters. Suitable promoters for use with mammalian host cells are well known and include those obtained from the genomes of viruses such as polyoma virus, fowlpox virus, adenovirus (such as Adenovirus 2), bovine papilloma virus, avian sarcoma virus, cytomegalovirus, a retrovirus, hepatitis-B virus and most preferably Simian Virus 40 (SV40). Other suitable mammalian promoters include heterologous mammalian promoters, e.g., heat-shock promoters and the actin promoter.

Additional promoters which may be used for expressing the selective binding agents of the invention include, but are not limited to: the SV40 early promoter region (Bernoist and Chambon, Nature, 290:304-310, 1981); the CMV promoter; the promoter contained in the 3′ long terminal repeat of Rous sarcoma virus (Yamamoto et al. (1980), Cell 22: 787-97); the herpes thymidine kinase promoter (Wagner et al. (1981), Proc. Natl. Acad. Sci. U.S.A. 78: 1444-5); the regulatory sequences of the metallothionine gene (Brinster et al, Nature, 296; 39-42, 1982); prokaryotic expression vectors such as the beta-lactamase promoter (Villa-Kamaroff, et al., Proc. Natl. Acad. Sci. U.S.A., 75; 3727-3731, 1978); or the tac promoter (DeBoer, et al. (1983), Proc. Natl. Acad. Sci. U.S.A., 80: 21-5). Also of interest are the following animal transcriptional control regions, which exhibit tissue specificity and have been utilized in transgenic animals: the elastase I gene control region which is active in pancreatic acinar cells (Swift et al. (1984), Cell 38: 639-46; Ornitz et al. (1986), Cold Spring Harbor Symp. Quant. Biol. 50: 399-409; MacDonald (1987), Hepatology 7: 425-515); the insulin gene control region which is active in pancreatic beta cells (Hanahan (1985), Nature 315: 115-22); the immunoglobulin gene control region which is active in lymphoid cells (Grosschedl et al. (1984), Cell 38; 647-58; Adames et al. (1985), Nature 318; 533-8; Alexander et al. (1987), Mol. Cell. Biol. 7: 1436-44); the mouse mammary tumor virus control region which is active in testicular, breast, lymphoid and mast cells (Leder et al. (1986), Cell 45: 485-95), albumin gene control region which is active in liver (Pinkert et al. (1987), Genes and Devel. 1: 268-76); the alphafetoprotein gene control region which is active in liver (Krumlauf et al. (1985), Mol. Cell. Biol. 5: 1639-48; Hammer et al. (1987), Science, 235: 53-8); the alpha 1-antitrypsin gene control region which is active in the liver (Kelsey et al. (1987), Genes and Devel. 1: 161-71); the beta-globin gene control region which is active in myeloid cells (Mogram et al., Nature, 315 338-340, 1985; Kollias et al. (1986), Cell 46: 89-94); the myelin basic protein gene control region which is active in oligodendrocyte cells in the brain (Readhead et al. (1987), Cell, 48: 703-12); the myosin light chain-2 gene control region which is active in skeletal muscle (Sani (1985), Nature, 314: 283-6); and the gonadotropic releasing hormone gene control region which is active in the hypothalamus (Mason et al. (1986), Science 234: 1372-8).

An enhancer sequence may be inserted into the vector to increase transcription in eucaryotic host cells. Several enhancer sequences available from mammalian genes are known (e.g., globin, elastase, albumin, alpha-feto-protein and insulin). Typically, however, an enhancer from a virus will be used. The SV40 enhancer, the cytomegalovirus early promoter enhancer, the polyoma enhancer, and adenovirus enhancers are exemplary enhancing elements for the activation of eukaryotic promoters.

While an enhancer may be spliced into the vector at a position 5′ or 3′ to the polypeptide coding region, it is typically located at a site 5′ from the promoter.

Preferred vectors for practicing this invention are those which are compatible with bacterial, insect, and mammalian host cells. Such vectors include, inter alia, pCRII, pCR3, and pcDNA3.1 (Invitrogen Company, San Diego, Calif.), pBSII (Stratagene Company, La Jolla, Calif.), pET15 (Novagen, Madison, Wis.), pGEX (Pharmacia Biotech, Piscataway, N.J.), pEGFP-N2 (Clontech, Palo Alto, Calif.), pETL (BlueBacII; Invitrogen), pDSR-alpha (PCT Publication No. WO90/14363) and pFastBacDual (Gibco/BRL, Grand Island, N.Y.).

Additional possible vectors include, but are not limited to, cosmids, plasmids or modified viruses, but the vector system must be compatible with the selected host cell. Such vectors include, but are not limited to plasmids such as Bluescript® plasmid derivatives (a high copy number ColE1-based phagemid, Stratagene Cloning Systems Inc., La Jolla Calif.), PCR cloning plasmids designed for cloning Taq-amplified PCR products (e.g., TOPO™ TA Cloning® Kit, PCR2.1 plasmid derivatives, Invitrogen, Carlsbad, Calif.), and mammalian, yeast or virus vectors such as a baculovirus expression system (pBacPAK plasmid derivatives, Clontech, Palo Alto, Calif.). The recombinant molecules can be introduced into host cells via transformation, transfection, infection, electroporation, or other known techniques.

Host cells of the invention may be prokaryotic host cells (such as E. coli) or eukaryotic host cells (such as a yeast cell, an insect cell, or a vertebrate cell). The host cell, when cultured under appropriate conditions, expresses an antibody or antigen binding domain of the invention which can subsequently be collected from the culture medium (if the host cell secretes it into the medium) or directly from the host cell producing it (if it is not secreted). Selection of an appropriate host cell will depend upon various factors, such as desired expression levels, polypeptide modifications that are desirable or necessary for activity, such as glycosylation or phosphorylation, and ease of folding into a biologically active molecule. A number of suitable host cells are known in the art and many are available from the American Type Culture Collection (ATCC), Manassas, Va. Examples include mammalian cells, such as Chinese hamster ovary cells (CHO) (ATCC No. CCL61) CHO DHFR-cells (Urlaub et al. Proc. Natl. Acad. Sci. USA 97, 4216-4220 (1980)), human embryonic kidney (HEK) 293 or 293T cells (ATCC No. CRL1573), or 3T3 cells (ATCC No. CCL92). The selection of suitable mammalian host cells and methods for transformation, culture, amplification, screening and product production and purification are known in the art. Other suitable mammalian cell lines, are the monkey COS-1 (ATCC No. CRL1650) and COS-7 cell lines (ATCC No. CRL1651), and the CV-1 cell line (ATCC No. CCL70). Further exemplary mammalian host cells include primate cell lines and rodent cell lines, including transformed cell lines. Normal diploid cells, cell strains derived from in vitro culture of primary tissue, as well as primary explants, are also suitable. Candidate cells may be genotypically deficient in the selection gene, or may contain a dominantly acting selection gene. Other suitable mammalian cell lines include but are not limited to, mouse neuroblastoma N2A cells, HeLa, mouse L-929 cells, 3T3 lines derived from Swiss, Balb-c or NIH mice, BHK or HaK hamster cell lines, which are available from the American Type Culture Collection, Manassas, Va.). Each of these cell lines is known by and available to those skilled in the art of protein expression.

Similarly useful as host cells suitable for the present invention are bacterial cells. For example, the various strains of E. coli (e.g., HB101, (ATCC No. 33694) DH5α, DH10, and MC1061 (ATCC No. 53338)) are well-known as host cells in the field of biotechnology. Various strains of B. subtilis, Pseudomonas spp., other Bacillus spp., Streptomyces spp., and the like may also be employed in this method.

Many strains of yeast cells known to those skilled in the art are also available as host cells for expression of the polypeptides of the present invention. Preferred yeast cells include, for example, Saccharomyces cerivisae.

Additionally, where desired, insect cell systems may be utilized in the methods of the present invention. Such systems are described for example in Kitts et al. (Biotechniques, 14, 810-817 (1993)), Lucklow (Curr. Opin. Biotechnol., 4, 564-572 (1993) and Lucklow et al. (J. Virol., 67, 4566-4579 (1993)). Preferred insect cells are Sf-9 and Hi5 (Invitrogen, Carlsbad, Calif.).

Transformation or transfection of a nucleic acid molecule encoding an anti-IL-1 antibody or antigen binding domain into a selected host cell may be accomplished by well known methods including methods such as calcium chloride, electroporation, microinjection, lipofection or the DEAE-dextran method. The method selected will in part be a function of the type of host cell to be used. These methods and other suitable methods are well known to the skilled artisan, and are set forth, for example, in Sambrook et al. supra.

One may also use transgenic animals to express glycosylated selective binding agents, such as antibodies and antigen binding domain. For example, one may use a transgenic milk-producing animal (a cow or goat, for example) and obtain glycosylated binding agents in the animal milk. Alternatively, one may use plants to produce glycosylated selective binding agents.

Host cells comprising (i.e., transformed or transfected) an expression vector encoding a selective binding agent of IL-1 may be cultured using standard media well known to the skilled artisan. The media will usually contain all nutrients necessary for the growth and survival of the cells. Suitable media for culturing E. coli cells are for example, Luria Broth (LB) and/or Terrific Broth (TB). Suitable media for culturing eukaryotic cells are RPMI 1640, MEM, DMEM, all of which may be supplemented with serum and/or growth factors as required by the particular cell line being cultured. A suitable medium for insect cultures is Grace's medium supplemented with yeastolate, lactalbumin hydrolysate, and/or fetal calf serum as necessary.

Typically, an antibiotic or other compound useful for selective growth of transfected or transformed cells is added as a supplement to the media. The compound to be used will be dictated by the selectable marker element present on the plasmid with which the host cell was transformed. For example, where the selectable marker element is kanamycin resistance, the compound added to the culture medium will be kanamycin. Other compounds for selective growth include ampicillin, tetracycline and neomycin.

The amount of an anti-IL-1 antibody or antigen binding domain produced by a host cell can be evaluated using standard methods known in the art. Such methods include, without limitation, Western blot analysis, SDS-polyacrylamide gel electrophoresis, non-denaturing gel electrophoresis, HPLC separation, immunoprecipitation, and/or activity assays.

Purification of an anti-IL-1 antibody or antigen binding domain which has been secreted into the cell media can be accomplished using a variety of techniques including affinity, immunoaffinity or ion exchange chromatography, molecular sieve chromatography, preparative gel electrophoresis or isoelectric focusing, chromatofocusing, and high pressure liquid chromatography. For example, antibodies comprising a Fc region may be conveniently purified by affinity chromatography with Protein A, which selectively binds the Fc region. Modified forms of an antibody or antigen binding domain may be prepared with affinity tags, such as hexahistidine or other small peptide such as FLAG (Eastman Kodak Co., New Haven, Conn.) or myc (Invitrogen) at either its carboxyl or amino terminus and purified by a one-step affinity column. For example, polyhistidine binds with great affinity and specificity to nickel, thus an affinity column of nickel (such as the Qiagen® nickel columns) can be used for purification of polyhistidine-tagged selective binding agents. (See for example, Ausubel et al, eds., Current Protocols in Molecular Biology, Section 10.11.8, John Wiley & Sons, New York (1993)). In some instances, more than one purification step may be required.

Selective binding agents of the invention which are expressed in procaryotic host cells may be present in soluble form either in the periplasmic space or in the cytoplasm or in an insoluble form as part of intracellular inclusion bodies. Selective binding agents can be extracted from the host cell using any standard technique known to the skilled artisan. For example, the host cells can be lysed to release the contents of the periplasm/cytoplasm by French press, homogenization, and/or sonication followed by centrifugation.

Soluble forms of an anti-IL-1 antibody or antigen binding domain present either in the cytoplasm or released from the periplasmic space may be further purified using methods known in the art, for example Fab fragments are released from the bacterial periplasmic space by osmotic shock techniques.

If an antibody or antigen binding domain has formed inclusion bodies, they can often bind to the inner and/or outer cellular membranes and thus will be found primarily in the pellet material after centrifugation. The pellet material can then be treated at pH extremes or with chaotropic agent such as a detergent, guanidine, guanidine derivatives, urea, or urea derivatives in the presence of a reducing agent such as dithiothreitol at alkaline pH or tris carboxyethyl phosphine at acid pH to release, break apart, and solubilize the inclusion bodies. The soluble selective binding agent can then be analyzed using gel electrophoresis, immunoprecipitation or the like. If it is desired to isolate a solublized antibody or antigen binding domain, isolation may be accomplished using standard methods such as those set forth below and in Marston et al. (Meth. Enz., 182:264-275 (1990)).

In some cases, an antibody or antigen binding domain may not be biologically active upon isolation. Various methods for “refolding” or converting the polypeptide to its tertiary structure and generating disulfide linkages, can be used to restore biological activity. Such methods include exposing the solubilized polypeptide to a pH usually above 7 and in the presence of a particular concentration of a chaotrope. The selection of chaotrope is very similar to the choices used for inclusion body solubilization, but usually the chaotrope is used at a lower concentration and is not necessarily the same as chaotropes used for the solubilization. In most cases the refolding/oxidation solution will also contain a reducing agent or the reducing agent plus its oxidized form in a specific ratio to generate a particular redox potential allowing for disulfide shuffling to occur in the formation of the protein's cysteine bridge(s). Some of the commonly used redox couples include cysteine/cystamine, glutathione (GSH)/dithiobis GSH, cupric chloride, dithiothreitol(DTT)/dithiane DTT, and 2-mercaptoethanol(bME)/dithio-b(ME). In many instances, a cosolvent may be used or may be needed to increase the efficiency of the refolding and the more common reagents used for this purpose include glycerol, polyethylene glycol of various molecular weights, arginine and the like.

Antibodies and antigen binding domains of the invention may also be prepared by chemical synthesis methods (such as solid phase peptide synthesis) using techniques known in the art such as those set forth by Merrifield et al. (1963), J. Am. Chem. Soc., 85: 2149, Houghten et al. (1985), Proc Natl Acad. Sci. USA, 82: 5132, and Stewart and Young (1984), Solid Phase Peptide Synthesis, Pierce Chemical Co., Rockford, Ill. Such polypeptides may be synthesized with or without a methionine on the amino terminus. Chemically synthesized antibodies and antigen binding domains may be oxidized using methods set forth in these references to form disulfide bridges. Antibodies so prepared will retain at least one biological activity associated with a native or recombinantly produced anti-IL-1 antibody or antigen binding domain.

Assays for Selective Binding Agents of IL-1

Screening methods for identifying selective binding agents that act by the method claimed are also part of the invention. For purposes of this invention, inhibiting the biological activity of IL-1 includes, but is not limited to, allowing binding of IL-1 to its cognate receptor but inhibiting receptor activation. An exemplary selective binding agent will prevent association of IL-1 and/or IL-1 receptor with IL-1 RAcP, thus inhibiting receptor activation in vivo by IL-1. Selective binding agents of the invention include anti-IL-1 antibodies, and fragments, variants, derivatives and fusion thereof, peptides, peptidomimetic compounds or organo-mimetic compounds.

Screening methods for identifying selective binding agents having the characteristics of the present invention can include in vitro or in vivo assays. In vitro assays include those that detect binding of IL-1 to IL-1R1 and IL-1RAcP to IL-1R1 and may be used to screen selective binding agents of IL-1 for their ability to increase or decrease the rate or extent of IL-1 binding to IL-1R1 and IL-1RAcP binding to IL-1R1. In one type of assay, an IL-1R1 polypeptide is immobilized on a solid support (e.g., agarose or acrylic beads) and a test ligand (e.g., IL-1 polypeptide, which may include a detectable label) is added either in the presence or absence of a selective binding agent for IL-1. The extent of binding of IL-1 to IL-1R1 with or without a selective binding agent present is measured. Binding can be detected by such techniques as radioactive labeling, fluorescent labeling or enzymatic reaction. Alternatively, the binding reaction may be carried out using a surface plasmon resonance detector system such as the BlAcore assay system (Pharmacia, Piscataway, N.J.). Binding reactions may be carried out according to the manufacturer's protocol.

In conjunction with these IL-1-IL-1R1 binding assays, the same selective binding agent candidates can be screened for their effect on IL-1R1-IL-1RAcP binding. In such assays, an IL-1RAcP polypeptide is used as the test ligand in place of the IL-1 polypeptide.

In vitro assays such as those described above may be used advantageously to screen rapidly large numbers of selective binding agents for effects on binding of IL-1 to IL-1R1. The assays may be automated to screen compounds generated in phage display, synthetic peptide and chemical synthesis libraries.

Selective binding agents increase or decrease activation of IL-1R1 by IL-1 and may also be screened in cell culture using cells and cell lines that functionally express IL-1R1. Cells and cell lines may be obtained from any mammal, but preferably will be from human or other primate, canine, or rodent sources. As an example, the activation of IL-1R1 is evaluated in the presence or absence of selective binding agents.

In vitro activity assays may also be used to identify selective binding agents which inhibit IL-1R1 activation. Examples of assays include stimulation of protein expression that is dependent on IL-1 activation of IL-1R1. In vivo assays are also available to determine whether a selective binding agent is capable of inhibiting activation of IL-1R1.

For diagnostic applications, in certain embodiments, selective binding agents of IL-1, such as antibodies and antigen binding domains thereof, typically will be labeled with a detectable moiety. The detectable moiety can be any one which is capable of producing, either directly or indirectly, a detectable signal. For example, the detectable moiety may be a radioisotope, such as ³H, ¹⁴C, ³²P, ³⁵S, or ¹²⁵I, a fluorescent or chemiluminescent compound, such as fluorescein isothiocyanate, rhodamine, or luciferin; or an enzyme, such as alkaline phosphatase, β-galactosidase or horseradish peroxidase. Bayer et al., Meth. Enz., 184: 138-163 (1990).

The selective binding agents of the invention may be employed in any known assay method, such as radioimmunoassays, competitive binding assays, direct and indirect sandwich assays (ELISAs), and immunoprecipitation assays (Sola, Monoclonal Antibodies: A Manual of Techniques, pp. 147-158 (CRC Press, 1987)) for detection and quantitation of IL-1 polypeptides. The antibodies will bind IL-1 polypeptides with an affinity which is appropriate for the assay method being employed.

The selective binding agents of the invention also are useful for in vivo imaging, wherein for example a selective binding agent labeled with a detectable moiety is administered to an animal, preferably into the bloodstream, and the presence and location of the labeled antibody in the host is assayed. The agent may be labeled with any moiety that is detectable in an animal, whether by nuclear magnetic resonance, radiology, or other detection means known in the art.

The invention also relates to a kit comprising a selective binding agent of IL-1, such as an antibody or antigen binding domain, and other reagents useful for detecting IL-1 levels in biological samples. Such reagents may include a secondary activity, a detectable label, blocking serum, positive and negative control samples, and detection reagents.

Therapeutic Methods of Use of IL-1 Selective Binding Agents

A disease or medical condition is considered to be an “interleukin-1 mediated disease” if the spontaneous or experimental disease or medical condition is associated with elevated levels of IL-1 in bodily fluids or tissue or if cells or tissues taken from the body produce elevated levels of IL-1 in culture. In many cases, such IL-1 mediated diseases are also recognized by the following additional two conditions: (1) pathological findings associated with the disease or medical condition can be mimicked experimentally in animals by administration of IL-1 or upregulation of expression of IL-1; and (2) a pathology induced in experimental animal models of the disease or medical condition can be inhibited or abolished by treatment with agents that inhibit the action of IL-1. In most IL-1 mediated diseases at least two of the three conditions are met, and in many IL-1 mediated diseases all three conditions are met.

A non-exclusive list of acute and chronic IL-1-mediated diseases includes but is not limited to the following:

acute pancreatitis;

ALS;

Alzheimer's disease;

cachexia/anorexia, including AIDS-induced cachexia;

asthma and other pulmonary diseases; atherosclerosis;

autoimmune vasculitis;

chronic fatigue syndrome;

Clostridium associated illnesses, including Clostridium-associated diarrhea;

coronary conditions and indications, including congestive heart failure, coronary restenosis, myocardial infarction, myocardial dysfunction (e.g., related to sepsis), and coronary artery bypass graft;

cancer, such as multiple myeloma and myelogenous (e.g., AML and CML) and other leukemias, as well as tumor metastasis;

diabetes (e.g., insulin diabetes);

endometriosis;

fever;

fibromyalgia;

glomerulonephritis;

graft versus host disease/transplant rejection;

hemohorragic shock;

hyperalgesia;

inflammatory bowel disease;

inflammatory conditions of a joint, including osteoarthritis, psoriatic arthritis and rheumatoid arthritis;

inflammatory eye disease, as may be associated with, for example, corneal transplant;

ischemia, including cerebral ischemia (e.g., brain injury as a result of trauma, epilepsy, hemorrhage or stroke, each of which may lead to neurodegeneration);

Kawasaki's disease;

learning impairment;

lung diseases (e.g., ARDS);

multiple sclerosis;

myopathies (e.g., muscle protein metabolism, esp. in sepsis);

neurotoxicity (e.g., as induced by HIV);

osteoporosis;

pain, including cancer-related pain;

Parkinson's disease;

periodontal disease;

pre-term labor;

psoriasis;

reperfusion injury;

septic shock;

side effects from radiation therapy;

temporal mandibular joint disease;

sleep disturbance;

uveitis;

or an inflammatory condition resulting from strain, sprain, cartilage damage, trauma, orthopedic surgery, infection or other disease processes.

Pharmaceutical Compositions

Pharmaceutical compositions of IL-1 selective binding agents are within the scope of the present invention. Such compositions comprise a therapeutically or prophylactically effective amount of an IL-1 selective binding agent such as an antibody, or a fragment, variant, derivative or fusion thereof, in admixture with a pharmaceutically acceptable agent. In a preferred embodiment, pharmaceutical compositions comprise an IL-1 selective binding agent in admixture with a pharmaceutically acceptable agent. Typically, the antibodies will be sufficiently purified for administration to an animal.

Pharmaceutically acceptable agents for use in the compositions of the invention include carriers, excipients, diluents, antioxidants, preservatives, coloring, flavoring and diluting agents, emulsifying agents, suspending agents, solvents, fillers, bulking agents, buffers, delivery vehicles, tonicity agents, cosolvents, wetting agents, complexing agents, buffering agents, antimicrobials and surfactants, as are well known in the art.

Neutral buffered saline or saline mixed with serum albumin are exemplary appropriate carriers. Also included in the compositions are antioxidants such as ascorbic acid; low molecular weight polypeptides; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as Tween, pluronics or polyethylene glycol (PEG). Also by way of example, suitable tonicity enhancing agents include alkali metal halides (preferably sodium or potassium chloride), mannitol, sorbitol and the like. Suitable preservatives include, but are not limited to, benzalkonium chloride, thimerosal, phenethyl alcohol, methylparaben, propylparaben, chlorhexidine, sorbic acid and the like. Hydrogen peroxide may also be used as preservative. Suitable cosolvents are for example glycerin, propylene glycol, and PEG. Suitable complexing agents are for example caffeine, polyvinylpyrrolidone, beta-cyclodextrin or hydroxy-propyl-beta-cyclodextrin. Suitable surfactants or wetting agents include sorbitan esters, polysorbates such as polysorbate 80, tromethamine, lecithin, cholesterol, tyloxapal and the like. The buffers can be conventional buffers such as acetate, borate, citrate, phosphate, bicarbonate, or Tris-HCl. Acetate buffer may be around pH 4.0-5.5 and Tris buffer may be around pH 7.0-8.5. Additional pharmaceutical agents are set forth in Remington's Pharmaceutical Sciences, 18th Edition, A. R. Gennaro, ed., Mack Publishing Company 1990, the relevant portions of which are hereby incorporated by reference.

The compositions may be in liquid form or in a lyophilized or freeze-dried form. Lypophilized forms may include excipients such as sucrose.

The compositions of the invention are suitable for parenteral administration. In preferred embodiments, the compositions are suitable for injection or infusion into an animal by any route available to the skilled worker, such as subcutaneous, intravenous, intramuscular, intraperitoneal, intracerebral (intraparenchymal), intracerebroventricular, intramuscular, intraocular, intraarterial, or intralesional routes. A parenteral formulation will typically be a sterile, pyrogen-free, isotonic aqueous solution, optionally containing pharmaceutically acceptable preservatives.

The optimal pharmaceutical formulation may be readily determined by one skilled in the art depending upon the intended route of administration, delivery format and desired dosage.

Other formulations are also contemplated by the invention. The pharmaceutical compositions also may include particulate preparations of polymeric compounds such as polylactic acid, polyglycolic acid, etc. or the introduction of an IL-1 selective binding agent (such as an antibody) into liposomes. Hyaluronic acid may also be used, and this may have the effect of promoting sustained duration in the circulation. Pharmaceutical compositions also include the formulation of IL-1 selective binding agents (such as antibodies) with an agent, such as injectable microspheres, bio-erodible particles or beads, or liposomes, that provides for the controlled or sustained release of a selective binding agent which may then be delivered as a depot injection. Other suitable means for delivery include implantable delivery devices.

A pharmaceutical composition comprising and IL-1 selective binding agent (such as an antibody) may be formulated as a dry powder for inhalation. Such inhalation solutions may also be formulated in a liquefied propellant for aerosol delivery. In yet another formulation, solutions may be nebulized.

It is also contemplated that certain formulations containing IL-1 selective binding agents may be administered orally. Formulations administered in this fashion may be formulated with or without those carriers customarily used in the compounding of solid dosage forms such as tablets and capsules. For example, a capsule may be designed to release the active portion of the formulation at the point in the gastrointestinal tract when bioavailability is maximized and pre-systemic degradation is minimized. Additional agents may be included to facilitate absorption of a selective binding agent. Diluents, flavorings, low melting point waxes, vegetable oils, lubricants, suspending agents, tablet disintegrating agents, and binders may also be employed.

Another preparation may involve an effective quantity of an IL-1 selective binding agent in a mixture with non-toxic excipients which are suitable for the manufacture of tablets. By dissolving the tablets in sterile water, or another appropriate vehicle, solutions can be prepared in unit dose form. Suitable excipients include, but are not limited to, inert diluents, such as calcium carbonate, sodium carbonate or bicarbonate, lactose, or calcium phosphate; or binding agents, such as starch, gelatin, or acacia; or lubricating agents such as magnesium stearate, stearic acid, or talc.

Additional formulations will be evident to those skilled in the art, including formulations involving IL-1 selective binding agents in combination with one or more other therapeutic agents. Techniques for formulating a variety of other sustained- or controlled-delivery means, such as liposome carriers, bio-erodible microparticles or porous beads and depot injections, are also known to those skilled in the art. See, for example, the Supersaxo et al. description of controlled release porous polymeric microparticles for the delivery of pharmaceutical compositions (See WO 93/15722 (PCT/US93/00829) the disclosure of which is hereby incorporated by reference.

Regardless of the manner of administration, the specific dose may be calculated according to body weight, body surface area or organ size. Further refinement of the calculations necessary to determine the appropriate dosage for treatment involving each of the above mentioned formulations is routinely made by those of ordinary skill in the art and is within the ambit of tasks routinely performed by them. Appropriate dosages may be ascertained through use of appropriate dose-response data.

One may further administer the present pharmaceutical compositions by pulmonary administration, see, e.g., PCT WO94/20069, which discloses pulmonary delivery of chemically modified proteins, herein incorporated by reference. For pulmonary delivery, the particle size should be suitable for delivery to the distal lung. For example, the particle size may be from 1 μm to 5 μm, however, larger particles may be used, for example, if each particle is fairly porous.

Alternatively or additionally, the compositions may be administered locally via implantation into the affected area of a membrane, sponge, or other appropriate material on to which an OP an IL-1 selective binding agent has been absorbed or encapsulated. Where an implantation device is used, the device may be implanted into any suitable tissue or organ, and delivery of an IL-1 selective binding agent may be directly through the device via bolus, or via continuous administration, or via catheter using continuous infusion.

Pharmaceutical compositions of the invention may also be administered in a sustained release formulation or preparation. Suitable examples of sustained-release preparations include semipermeable polymer matrices in the form of shaped articles, e.g. films, or microcapsules. Sustained release matrices include polyesters, hydrogels, polylactides (See e.g., U.S. Pat. No. 3,773,919, EP 58,481), copolymers of L-glutamic acid and gamma ethyl-L-glutamate (Sidman et al. (1983), Biopolymers, 22: 547-556), poly (2-hydroxyethyl-methacrylate) (Langer et al. (1981), J. Biomed. Mater. Res., 15: 167-277 and Langer (1982), Chem. Tech., 12: 98-105), ethylene vinyl acetate, or poly-D(−)-3-hydroxybutyric acid. Sustained-release compositions also may include liposomes, which can be prepared by any of several methods known in the art. See e.g., Eppstein et al. (1985), Proc. Natl. Acad. Sci. USA 82: 3688-92; EP 36,676; EP 88,046; and EP 143,949.

IL-1 selective binding agents, such as antibodies and fragments, variants, derivatives and fusions thereof, may be employed alone or in combination with other pharmaceutical compositions. For example, pharmaceutical compositions comprising separately or together an IL-1 antagonist and an interleukin-1 receptor antagonist, or an IL-1 antagonist and a soluble TNF receptor-1, or an IL-1 antagonist and a soluble TNF receptor-2 (e.g., etanercept) or another TNF inhibitor (e.g., an antibody such as D2E7) may be used for the treatment of rheumatoid arthritis. Further, compositions comprising separately or together an IL-1 antagonist and a cancer therapy agent may be used for the treatment of cancer and associated inflammation and effects on bone mass. Other combinations with an IL-1 antagonist are possible depending upon the condition being treated.

It may be desirable in some instances to use a pharmaceutical composition comprising an IL-1 selective binding agent compositions in an ex vivo manner. Here, cells, tissues, or organs that have been removed from the patient are exposed to pharmaceutical compositons comprising IL-1 selective binding agents after which the cells, tissues and/or organs are subsequently implanted back into the patient.

In other cases, a composition comprising an IL-1 selective binding agent may be delivered through implanting into patients certain cells that have been genetically engineered, using methods such as those described herein, to express and secrete the polypeptides, selective binding agents, fragments, variants, or derivatives. Such cells may be animal or human cells, and may be derived from the patient's own tissue or from another source, either human or non-human. Optionally, the cells may be immortalized. However, in order to decrease the chance of an immunological response, it is preferred that the cells be encapsulated to avoid infiltration of surrounding tissues. The encapsulation materials are typically biocompatible, semi-permeable polymeric enclosures or membranes that allow release of the protein product(s) but prevent destruction of the cells by the patient's immune system or by other detrimental factors from the surrounding tissues.

Methods used for membrane encapsulation of cells are familiar to the skilled artisan, and preparation of encapsulated cells and their implantation in patients may be accomplished without undue experimentation. See, e.g., U.S. Pat. Nos. 4,892,538; 5,011,472; and 5,106,627. A system for encapsulating living cells is described in PCT WO 91/10425 (Aebischer et al.). Techniques for formulating a variety of other sustained or controlled delivery means, such as liposome carriers, bio-erodible particles or beads, are also known to those in the art, and are described. The cells, with or without encapsulation, may be implanted into suitable body tissues or organs of the patient.

A therapeutically or prophylactically effective amount of a pharmaceutical composition comprising an IL-1 selective binding agent (such as an anti-IL-1 antibody, or fragment, variant, derivative, and fusion thereof) will depend, for example, upon the therapeutic objectives such as the indication for which the composition is being used, the route of administration, and the condition of the subject. IL-1 antagonist antibodies or antigen binding domains of the invention are administered in a therapeutically or prophylactically effective amount to prevent and/or treat loss of bone associated with metastatic bone disease. A “therapeutically or prophylactically effective amount” of an IL-1 antagonist antibody is that amount which affects a parameter of an IL-1 mediated disease as defined hereinbefore.

Accordingly, it may be necessary for the caretaker to titer the dosage and modify the route of administration as required to obtain the optimal therapeutic effect. A typical dosage may range from about 0.1 μg/kg to up to about 100 mg/kg or more, depending on the factors mentioned above. In other embodiments, the dosage may range from 1 μg/kg up to about 100 mg/kg; or 5 μg/kg up to about 100 mg/kg; or 0.1 μg/kg up to about 100 mg/kg; or 1 μg/kg up to about 100 mg/kg Typically, a clinician will administer the composition until a dosage is reached that achieves the desired effect. The composition may therefore be administered as a single dose, or as two or more doses (which may or may not contain the same amount of an IL-1 selective binding agent) over time, or as a continuous infusion via implantation device or catheter.

### DETAILED DESCRIPTION OF PREFERRED EMBODIMENTS

### EXAMPLE 1

Methods

sIL-1RAcP Constructs

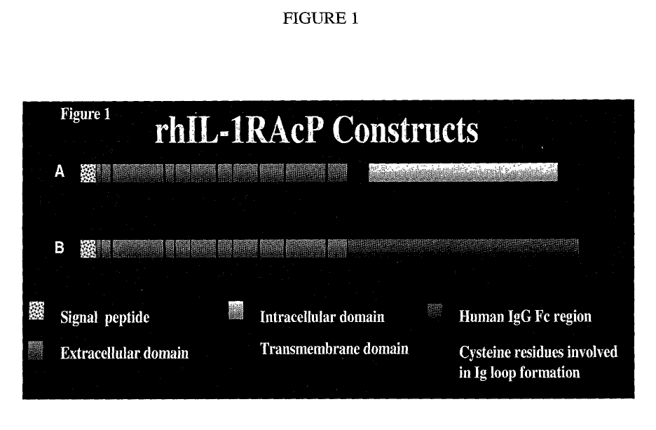

IL-1RAcP consists of an extracellular portion containing three Ig-like domains, a short transmembrane domain and an intracellular domain involved in IL-1• signal transduction (FIG. 1, A). A soluble, chimeric protein consisting of the complete extracellular domain of IL-1RAcP joined at it's c-terminus to human Fc (FIG. 1, B) was assembled in a mammalian expression vector. The recombinant protein was purified from the conditioned medium of a stable CHO cell line by FPLC using protein G affinity chromatography.

Synoviocyte Assay

The human synoviocyte cell line SW982 was seeded into 96-well plates at a density of 7500 cells/well. Cells were allowed to adhere (˜4 hrs) before treatment with 20 pM IL-1• (EC50) +/− inhibitors. Conditioned medium was harvested after 24 hrs and IL-6 production was quantified by ELISA.

HUVEC Assay

HUVEC cells were seeded into 96-well plates at a density of at 2×10⁴ cells/well 24 hrs prior to treatment. The cells were pre-treated with inhibitors for 30 minutes prior to stimulation with 7 pM.

IL-1β (EC50) for 18 hours. The conditioned medium was collected, diluted 1:100 and the GRO-alpha levels measured by ELIZA (R&D Systems).

In Vitro Inhibition of IL-1 Binding to IL-1R1

96-well ELISA plates were coated with a non receptor blocking anti-IL-1β antibody. IL-1β was captured onto the antibody coated plates. Inhibitors were titrated with sIL-1R1 added at KD. IL-1/IL-1R1 complex was detected with a non-neutralizing anti-IL-1R1 antibody (Pharmingen).

In Vitro Inhibition of IL-1RAcP Binding to IL-1 Bound IL-1R1

96-well ELISA plates were coated with sIL-1R1. Inhibitors were titrated vs. IL-1R1 with IL-1β added at KD and a molar excess of sIL-1RAcP-Fc. IL-1/IL-1R1/IL-1RAcP complex was detected with an HRP conjugated anti-Human Fc antibody (Pierce).

Calculation of Binding and Competition Parameters

Binding and competition curves were analyzed using PRIZM™ software with non-linear regression with one-site binding or one-site competition.

TABLE 1
Potency of IL-1 Inhibitors in a Synoviocyte Bioassay

| | Anti-IL-1β | Anti-IL-1β | | Anti-IL-1R1 | Anti-RAcP |
|---|---|---|---|---|---|
| | MAB 201 | AF 201 | | 4C1 | 8H1-1 |
| IC50 | (R&D) | (R&D) | IL-1ra | (BIOSOURCE) | (AMGEN) |
| Best-fit Values | 4.951 | 18.1 | 48.42 | 90.5 | 1528 |
| 95% | 3.404 to 7.202 | 11.83 to 27.70 | 28.70 to 81.68 | 126.8 to 286.5 | 773.0 to 3021 |
| Confidence | | | | | |
| Intervals | | | | | |

TABLE 2
Potency of IL-1 Inhibitors in HUVECs

| | Anti-IL- | Anti-IL- | | | | |
|---|---|---|---|---|---|---|
| | 1b | 1B | Anti-IL-1R1 | | Anti-RAcP | Anti-RAcP |
| | MAB201 | AF201 | 4C1 | | 8G2-6 | 8H1-12 |
| Best-fit values | (R&D) | (R&D) | (BIOSOURCE) | IL-1RA | (Amgen) | (Amgen) |
| IC50 (pM) | 171.4 | 843.4 | 30690 | 2930 | 131600 | 497700 |
| IC50 95% | 103.0 to | 299.8 to | 25560 to | 1816 to | 106400 to | 433000 to |
| Confidence | 285.5 | 2373 | 36850 | 4727 | 162800 | 572000 |
| Intervals | | | | | | |
| Goodness of Fit, | 0.9814 | 0.9253 | 0.9922 | 0.9789 | 0.9744 | 0.9792 |
| R² | | | | | | |

### EXAMPLE 2

### Preparation of Chimeric and Humanized Antibodies

Peptide Sequence Analysis:

The sequence of the MAB201 murine antibody was determined by protein sequencing (Edman degradation of peptides generated by various proteases) of the commercial R&D Systems product. The V regions of the mouse monoclonal MAB201 sequences are as follows:

Heavy chain V (SEQ ID NO:3):
QVTLKESGPGILKPSQTLSLTCSFSGFSLSTSGMGVGWIRQPSGKGLEWL
AHIWWDGDESYNPSLKTRLTISKNTSRNQVFLKTTTVDTVDTATYFCARN
RYDPPWFVDWGQGTLVTVSS
Kappa chain V (SEQ ID NO:4):
DTQMTQTTSSLSASLGDRVTISCRASQDISNYLSWYQQKPDGTVKLLIYY
TSKLHSGVPSRFSGSGSGTDYFLTISNLEQEDIATYFCLQGKMLPWTFGG
GTKLEIK

Gene Assembly:

Chimeric and CDR-grafted (humanized) antibody variable regions were constructed by a combination of shot-gun gene synthesis (Grundstrom et al, 1985) and the recursive PCR method of Prodromou and Pearl (1997).

a) Chimeric Antibody Construction

The gamma chain chimera was constructed by annealing and ligating the set of overlapping complementary oligonucleotides C through O (Table 3), followed by PCR amplification using flanking primers A and B. The product of this reaction was used as template in a PCR reaction with primers B and P which added a 3′ BsmBI restriction site.

A mixture containing 10 pmol of each of oligonucleotides C through 0 (Table 3) was combined in 1× T4 Ligase buffer (New England Biolabs), containing ATP, and heated to boiling in a water bath for 5 minutes, then allowed to slowly cool to room temperature. One microliter of high concentration T4 DNA Ligase (NEB®) was added and the reaction incubated at room temperature for 2 hours. One microliter of the ligation reaction was used as template in a PCR, performed using the following conditions: 2 cycles of incubations at 94° C. for 20 seconds, 55° C. for 30 seconds and 74° C. for 1 minute; followed by 25 cycles of incubations at 94° C. for 20 seconds, 60° C. for 30 seconds and 74° C. for 1 minute. The resulting PCR product was purified with a Qiagen® QIAquick® column, and then sequentially digested using restriction endonucleases XbaI and BsmBI. The XbaI-BsmBI fragment was ligated into the XbaI-BsmBI digested expression vector containing a human IgG1 constant region and a DHFR selection cassette (pDSRα19/hIgG1). This ligation results in a proper fusion of the V region to the constant region. The Vector contains SV40 promoter and aFSH poly A sequences.

The kappa chain chimera was constructed similarly using oligonucleotides iii through xv in Table 4, amplified by primers i and ii. The kappa chain variable region was cloned by digestion with XbaI and BsmBI into a human kappa constant region in pDSRa19/hCₖₐₚₚₐ. This ligation results in a proper fusion of the V region with the constant region.

TABLE 3

Oligonucleotides used to assemble murine gamma chain
variable region for chimeric construction

| | | |
|---|---|---|
| Primer A | CTT CTT CAC TCA CCA CCG TAT GG | SEQ ID NO:5 |
| Primer B | CCT TCT TCA TCC ACC GCA CG | SEQ ID NO:6 |
| Oligo C | CCC TGT CCC CAG TCC ACG AAC CAT GGA GGG TCG | SEQ ID NO:7 |
| | TAT CTG TTG CGG GCA CA | |
| Oligo D | CAG TGT CCA CTG TGG TGA TCT TGA GGA ACA CCT | SEQ ID NO:8 |
| | GGT TCC TGC TGG TGT TC | |
| Oligo E | CTT CAG GGA TGG GTT GTA GGA CTC GTC CCC GTC | SEQ ID NO:9 |
| | CCA CCA GAT GTG TGC AA | |
| Oligo F | GAT GGC TGT CTG ATC CAA CCC ACC CCC ATT CCG | SEQ ID NO:10 |
| | GAG GTA CTC AGT GAG AA | |
| Oligo G | GGG ACA GGG TCT GGC TGG GCT TCA GGA TTC CGG | SEQ ID NO:11 |
| | GAC CGG ACT CCT TCA GG | |
| Oligo H | TCT CAG CCA CAG CAG CAG GAG CCC CAG GAG CTG | SEQ ID NO:12 |
| | AGC GGG CAC CCT CAT GT | |
| Oligo I | pCA TGG TTC GTG GAC TGG GGA CAG GGA ACC CTG | SEQ ID NO:13 |
| | GTC ACC GTC TCT AGT CCA TAC GGT GGT GAG TGA | |
| | AGA AGG G | |
| Oligo J | pCT CAA GAT CAC CAC AGT GGA CAC TGT GGA CAC | SEQ ID NO:14 |
| | CGC CAC CTA CTT CTG TGC CCG CAA CAG ATA CGA | |
| | CCC TC | |
| Oligo K | pCG AGT CCT ACA ACC CAT CCC TGA AGA CCC GGC | SEQ ID NO:15 |
| | TGA CAA TCT CCA AGA ACA CCA GCA GGA ACC AGG | |
| | TGT TC | |
| Oligo L | pGG GTG GGT TGG ATC AGA CAG CCA TCC GGC AAG | SEQ ID NO:16 |
| | GGT CTG GAG TGG CTT GCA CAC ATC TGG TGG GAC | |
| | GGG GA | |
| Oligo M | pCT GAA GCC CAG CCA GAC CCT GTC CCT GAC CTG | SEQ ID NO:17 |
| | TAG CTT CTC TGG GTT CTC ACT GAG TAC CTC CGG | |
| | AAT GG | |
| Oligo N | pGG GGC TCC TGC TGC TGT GGC TGA GAG GTG CCA | SEQ ID NO:18 |
| | GAT GTC AGG TGA CCC TGA AGG AGT CCG GTC CCG | |
| | GAA TC | |
| Oligo O | CCT TCT TCA TCC ACC GCA CGG CAA CAA GCT TCT | SEQ ID NO:19 |
| | ACA CCA CCA TGG ACA TGA GGG TGC CCG CTC AGC | |
| | TCC T | |
| Oligo P | GTG GAG GCA CTA GAG ACG GTG ACC AGG GTT CC | SEQ ID NO:20 |

p = 5′ phosphorylated

TABLE 4

Oligonucleotides (sequences 5′ to 3′) used to assemble murine
kappa chain variable region for chimeric construction

| | | |
|---|---|---|
| Primer i | CAG GCG GTT CAT CTG GAG ATA G | SEQ ID NO:21 |
| Primer ii | CAT TCC TGG AGT GTC CAA GTG TG | SEQ ID NO:22 |
| Oligo iii | CGT CTC ACA GTT CTC TTG ATT TCC AGC TTA GTC | SEQ ID NO:23 |
| | CCG CCT CCG AAG GTC CA | |
| Oligo iv | GAC AGA AGT ATG TGG CAA TGT CCT CCT GCT CCA | SEQ ID NO:24 |
| | AGT TGC TGA TGG TCA GG | |
| Oligo v | AGA GCC GCT GAA CCG ACT AGG CAC GCC GGA GTG | SEQ ID NO:25 |
| | GAG CTT GCT GGT GTA AT | |
| Oligo vi | CCG TCG GGC TTC TGC TGG TAC CAA CTC AGG TAG | SEQ ID NO:26 |
| | TTG GAA ATG TCC TGG GA | |
| Oligo vii | CTC TGT CTC CCA GAG AGG CAC TCA GGG AGC TTG | SEQ ID NO:27 |
| | TGG TCT GGG TCA TCT GG | |
| Oligo viii | CAG CCA CAG CAG CAG GAG CCC CAG GAG CTG AGC | SEQ ID NO:28 |
| | GGG CAC CCT CAT GTC CA | |
| Oligo ix | pTG GAA ATC AAG AGA ACT GTG AGA CGT AAT GTC | SEQ ID NO:29 |
| | TAT CTC CAG ATG AAC CGC CTG | |
| Oligox | pGA GGA CAT TGC CAC ATA CTT CTG TCT GCA GGG | SEQ ID NO:30 |
| | GAA GAT GCT GCC CTG GAC CTT CGG AGG CGG GAC | |
| | TAA GC | |
| Oligo xi | pCG TGC CTA GTC GGT TCA GCG GCT CTG GAA GTG | SEQ ID NO:31 |
| | GCA CCG ACT ACT TCC TGA CCA TCA GCA ACT TGG | |
| | AGC AG | |
| Oligo xii | PGT TGG TAC CAG CAG AAG CCC GAC GGC ACC GTG | SEQ ID NO:32 |
| | AAG CTG CTC ATC TAT TAC ACC AGC AAG CTC CAC | |
| | TCC GG | |
| Oligo xiii | pCT GAG TGC CTC TCT GGG AGA CAG AGT GAC CAT | SEQ ID NO:33 |
| | CAG CTG TAG AGC CTC CCA GGA CAT TTC CAA CTA | |
| | CCT GA | |
| Oligo xiv | pCC TGG GGC TCC TGC TGC TGT GGC TGA GAG GTG | SEQ ID NO:34 |
| | CCA GAT GTG ACA TCC AGA TGA CCC AGA CCA CAA | |
| | GCT CC | |
| Oligo xv | CCA TTC CTG GAG TGT CCA AGT GTG CAA CAA GCT | SEQ ID NO:35 |
| | TCT AGA CCA CCA TGG ACA TGA GGG TGC CCG CTC | |
| | AGC T | |

p = 5′ phosphorylated

b) CDR Grafting (Antibody Humanization)

Variable regions of the mouse antibody sequences were compared and CDRs identified by aligning sequences against a database of human germline antibody variable regions (VBASE http://www.mrc-cpe.cam.ac.uk/imt-doc/restricted/ok.html) (Ian Tomlinson, MRC Centre for Protein Engineering, Hills Road, Cambridge CB2 2QH, U.K.). CDR definitions are followed as defined by Kabat (Kabat et al., Sequences of Immunological Interest, 5^{th} ed., U.S. Department of Health and Human Services, 1991).

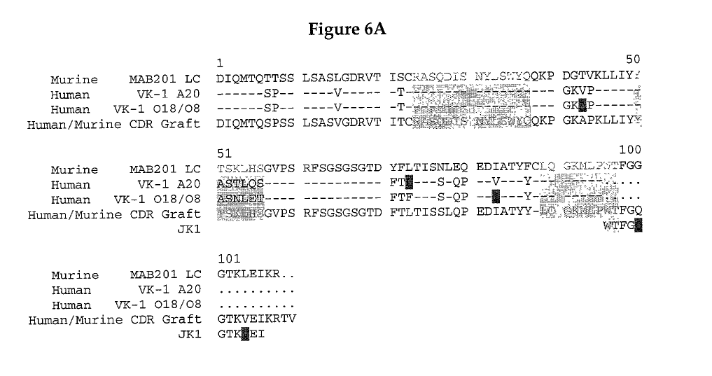

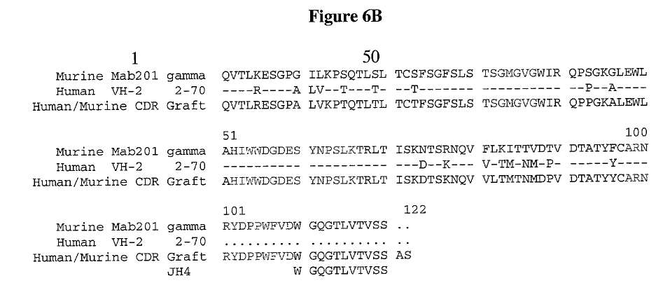

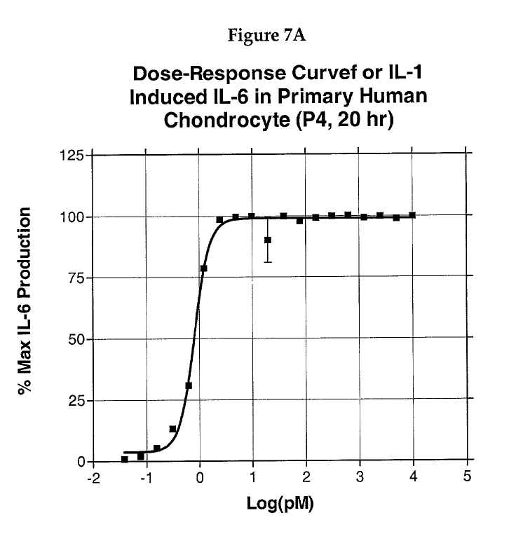

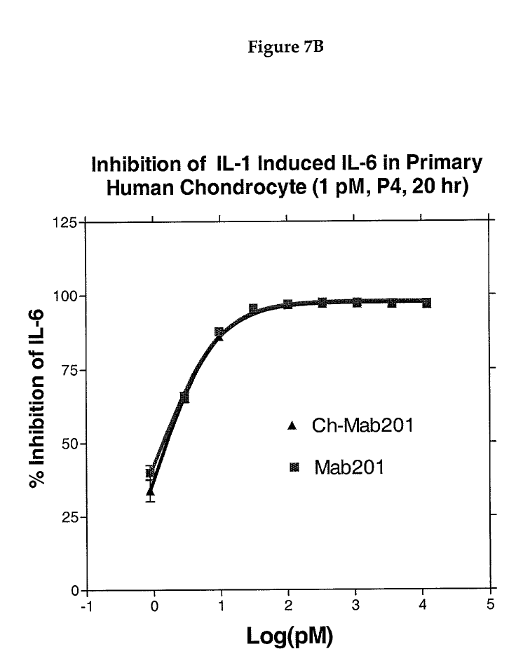

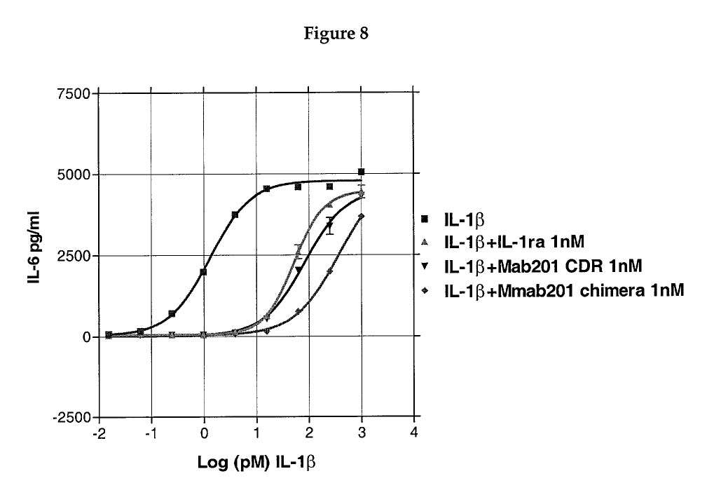

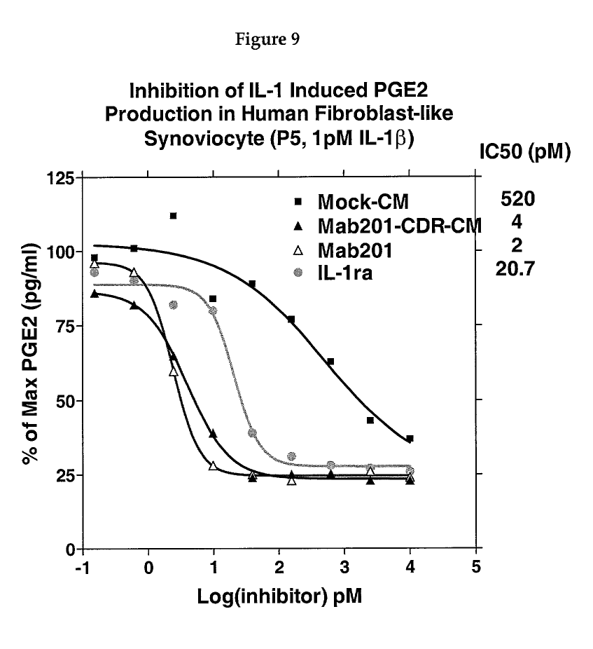

For the kappa chain, framework regions from two germline sequences were used as acceptor sequences onto which the murine CDRs were grafted. Germline region variable regions VK-1 A20 and VK-1 018/08 were utilized. The human JK1 joining region was determined to have the greatest homology to the mouse J region and the appropriate amino acid changes were made (see FIG. 6). In the framework and joining regions, murine amino acids were replaced with human amino acids at positions 7, 8, 15, 22, 41, 42, 43, 44, 71, 72, 73, 77, 79, 80, 83, 87, 100 and 104 (see FIG. 6).

PCR with oligonucleotides containing appropriate codon changes was used to create the humanized kappa chain. An existing human kappa chain clone (AT kappa), based on the human framework VK-1 O12/O2, was used as a template.

Twenty nanograms of AT kappa chain DNA were used as template for PCR with the following primer pairs. All PCR reactions were done using Pfu (Stratagene®) polymerase and were hot started by addition of the polymerase once the reaction temperature reached 94° C. The PCR was performed using the following conditions: 2 cycles of incubation at 94° C. for 20 seconds, 55° C. for 30 seconds and 74° C. for 1 minute; followed by 25 cycles of incubation at 94° C. for 20 seconds, 66° C. for 30 seconds and 74° C. for 1 minute. The resulting PCR-amplified products were punched from agarose gels using Pasteur pipettes and the agarose plug was boiled in 20 μl water for 5 minutes to release the DNA fragment. The resulting solution was used as the template for further extension or overlap PCR.

| | |
|---|---|
| Oligo 1 (+) | |
| GTG GTT GAG AGG TGC CAG ATG TGA CAT CCA GAT GAC CCA GTC T | (SEQ ID NO:36) |
| and oligo 2 (−) | |
| CCA GCT TAA ATA GTT GCT AAT GTC CTG ACT TGC CCG GCA | (SEQ ID NO:37) |
| were used to amplify product A. Oligo 3 (+) | |
| AAC TAT TTA AGC TGG TAT CAG CAG AAA CCA GGG AAA GCC | (SEQ ID NO:38) |
| and oligo 4 (−) | |
| GGT GTA ATA GAT CAG GAG CTT AGG GGC TTT CCC TGG TTT | (SEQ ID NO:39) |
| produced product B. Oligo 6 (+) | |
| TCC AAG TTG CAC AGT GGA GTC CCA TCA | (SEQ ID NO: 40) |
| and oligo 7 (−) | |
| GGT GAG AGT GAA GTC GGT CCC AGA TCC ACT GCC | (SEQ ID NO:41) |
| resulted in product C. Oligo 8 (+) | |
| AGT GGA TCT GGG ACC GAC TTC ACT CTC ACC ATC | (SEQ ID NO:42) |
| and oligo 9 (−) | |
| GGT CCA GGG CAG CAT CTT GCC CTG CAG ACA GTA GTA AGT GGC | (SEQ ID NO:43) |
| were used to generate product D. | |
| Oligos 3 and 5 (−) | |
| TCC ACT GTG CAA CTT GGA GGT GTA ATA GAT CAG | (SEQ ID NO:44) |
| were used to lengthen the 3′ end of product B. Oligo 8 and oligo 10 (−) | |
| CTT GGT CCC TTG GCC GAA GGT CCA GGG CAG CAT CTT | (SEQ ID NO:45) |
| were used to append sequence onto the 3′ end of product D. | |
| Products A and B were combined and amplified using | |
| oligonucleotides 1 and 11 (−) | |
| TGA TGG GAC TCC ACT GTG CAA CTT GGA | (SEQ ID NO:46) |
| to produce fragment E, while products C and D were combined and | |
| amplified by overlap PCR using oligos 6 and 12 (−) | |
| ATT ACG TCT CAC AGT TCG TTT GAT TTC CAC CTT GGT CCC TTG GCC GAA | (SEQ ID NO:47) |
| and resulted in product F. Products E and F were isolated from agarose | |
| gels and extended by overlap PCR with oligo 13 (+) | |
| CCG CTC AGC TCC TGG GGC TCC TGC TAT TGT GGT TGA GAG GTG CCA GAT | (SEQ ID NO:48 |
| and 12 followed by sequential PCR with oligo 14(+) | |
| CAG CAG AAG CTT CTA GAC CAC CAT GGA CAT GAG GGT CCC CCC TCA GCT CCT GGG | (SEQ ID NO:49 |

and 12. The resulting final product was purified by Qiagen® column and digested with XbaI and BsmBI and ligated into complementary sites in a human kappa chain constant region vector pDSRa19/hCₖₐₚₚₐ.

TABLE 5

Oligonucleotides used to generate the CDR grafted MAB201
kappa chain.

| | | |
|---|---|---|
| Oligo 1 | GTG GTT GAG AGG TGC CAG ATG TGA CAT CCA GAT GAC CCA GTC T | SEQ ID NO:50 |
| Oligo2 | CCA GCT TAA ATA GTT GCT AAT GTC CTG ACT TGC CCG GCA | SEQ ID NO:51 |
| Oligo 3 | AAC TAT TTA AGC TGG TAT CAG CAG AAA CCA GGG AAA GCC | SEQ ID NO:52 |
| Oligo 4 | GGT GTA ATA GAT CAG GAG CTT AGG GGC TTT CCC TGG TTT | SEQ ID NO:53 |
| Oligo 5 | TCC ACT GTG CAA CTT GGA GGT GTA ATA GAT CAG | SEQ ID NO:54 |
| Oligo 6 | TCC AAG TTG CAC AGT GGA GTC CCA TCA | SEQ ID NO:55 |
| Oligo 7 | GGT GAG AGT GAA GTC GGT CCC AGA TCC ACT GCC | SEQ ID NO:56 |
| Oligo 8 | AGT GGA TCT GGG ACC GAC TTC ACT CTC ACC ATC | SEQ ID NO:57 |
| Oligo 9 | GGT CCA GGG CAG CAT CTT GCC CTG CAG ACA GTA GTA AGT GGC | SEQ ID NO:58 |
| Oligo 10 | CTT GGT CCC TTG GCC GAA GGT CCA GGG CAG CAT CTT | SEQ ID NO:59 |
| Oligo 11 | TGA TGG GAC TCC ACT GTG CAA CTT GGA | SEQ ID NO:60 |
| Oligo 12 | ATT ACG TCT CAC AGT TCG TTT GAT TTC CAC CTT GGT CCC TTG GCC GAA | SEQ ID NO:61 |
| Oligo 13 | CCG CTC AGC TCC TGG GGC TCC TGC TAT TGT GGT TGA GAG GTG CCA GAT | SEQ ID NO:62 |
| Oligo 14 | CAG CAG AAG CTT CTA GAC CAC CAT GGA CAT GAG GGT CCC CGC TCA GCT CCT GGG | SEQ ID NO:63 |

For the gamma chain, variable region VH-2, locus 2-70 was determined to be the closest in identity in the framework and CDR regions and was chosen as the humanization framework. Murine amino acids at positions 5, 10, 11, 12, 15, 19, 23, 43, 46, 74, 77, 81, 83, 84, 86, 87, 89 and 96 were replaced with human residues (see FIG. 6).

TABLE 6

Oligonucleotides used to generate the CDR grafted MAB201
gamma chain.

| | | |
|---|---|---|
| Oligo 15 | GGA TGG AGA TTG GGA TGG AAA TGG CAG CAG AAG CTT | SEQ ID NO:64 |
| | CTA GAC CAC CAT GGA CAT GAG GGT CCC CGC TCA G | |
| Oligo 16 | CTC CTG GGG CTC CTG CTA TTG TGG TTG AGA GGT GCC | SEQ ID NO:65 |
| | AGA TGT CAG GTC ACC TTG AGG GAG TCT GGT CCT G | |
| Oligo 17 | CGC TGG TGA AAC CCA CAC AGA CCC TCA CAC TGA CCT | SEQ ID NO:66 |
| | GCA CCT TCT CTG GGT TCT CAC TCA GCA CTA GTG G | |
| Oligo 18 | AAT GGG CGT GGG CTG GAT GCG TCA GCC CCC AGG GAA | SEQ ID NO:67 |
| | GGC CCT GGA GTG GCT TGC ACA CAT TTG GTG GGA C | |
| Oligo 19 | GGT GAT GAG TCC TAC AAC CCA TCT CTG AAG ACC AGG | SEQ ID NO:68 |
| | CTC ACC ATC TCC AAG GAC ACC TCC AAA AAC CAG G | |
| Oligo 20 | TGG TCC TTA CAA TGA CCA ACA TGG ACC CTG TGG ACA | SEQ ID NO:69 |
| | CAG CCA CGT ATT ATT GTG CAC GGA ACA GGT ACG A | |
| Oligo 21 | CCC ACC TTG GTT CGT GGA CTG GGG CCA GGG CAC CCT | SEQ ID NO:70 |
| | GGT CAC CGT CTC TAG TGC CTC CAC GCT GTT CTT | |
| | TCC CTG AAT CTC GCC | |
| Oligo 22 | ACC ACA ATA GCA GGA GCC CCA GGA GCT GAG CGG GGA | SEQ ID NO:71 |
| | CCC TCA TGT CCA TG | |
| Oligo 23 | AGG GTC TGT GTG GGT TTC ACC AGC GCA GGA CCA GAC | SEQ ID NO:72 |
| TCC CTC AAG GTG AC | | |
| Oligo 24 | CTG ACG GAT CCA GCC CAC GCC CAT TCC ACT AGT GCT | SEQ ID NO:73 |
| | GAG TGA GAA CCC AG | |
| Oligo 25 | GAG ATG GGT TGT AGG ACT CAT CAC CGT CCC ACC AAA | SEQ ID NO:74 |
| | TGT GTG CAA GCC AC | |
| Oligo 26 | TCC AAG GAC ACC TCC AAA AAC CAG GTG GTC CTT ACA | SEQ ID NO:75 |
| | ATG ACC AAC ATG GA | |
| Oligo 27 | GCC CCA GTC CAC GAA CCA AGG TGG GTC GTA CCT GTT | SEQ ID NO:76 |
| | CCG TGC ACA ATA AT | |
| Oligo 28 | GGA TGG AGA TTG GGA TGG AAA TGC | SEQ ID NO:77 |
| Oligo 29 | AGG GAA AGA ACA GCG TGG AGG C | SEQ ID NO:78 |

Antibody Expression

The circular plasmids were used for transient expression in 293T cells. The serum free harvest from a 10 cm plate was used for affinity determination and activity assay. Once the chimeric antibody showed as active as the mouse monoclonal MAB201, stable cell lines were then generated.

The purified, linearized DNA was introduced into a CHOd-cell line using the Calcium Phosphate Transfection Kit (Invitrogen) as indicated by the manufacturer. Colonies were pooled and expression was done in roller bottles.

Antibody Purification and Affinity Measurements:

Chimeric and CDR grafted antibodies were purified by protein A column. The expression levels for both chimeric and CDR grafted antibodies were at approximately 2 μg/ml. The chimeric antibody displayed two bands for the heavy chain as analyzed by a reducing gel, indicating a possible partial glycosylation on heavy chain. This was later confirmed by LC/MS analysis to be a partial glycosylation at the Fv region of the heavy chain. For the CDR grafted MAb201, this glycosylation site was removed and thus the CDR grafted molecule does not show two bands in a reducing gel.

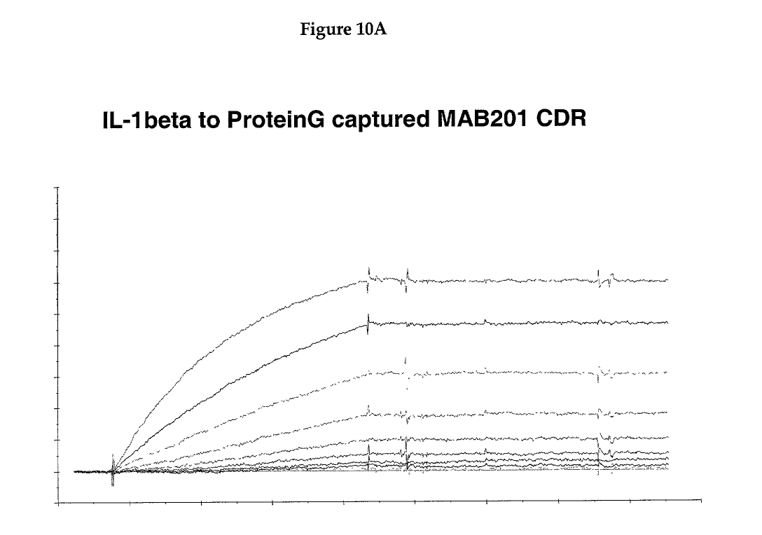

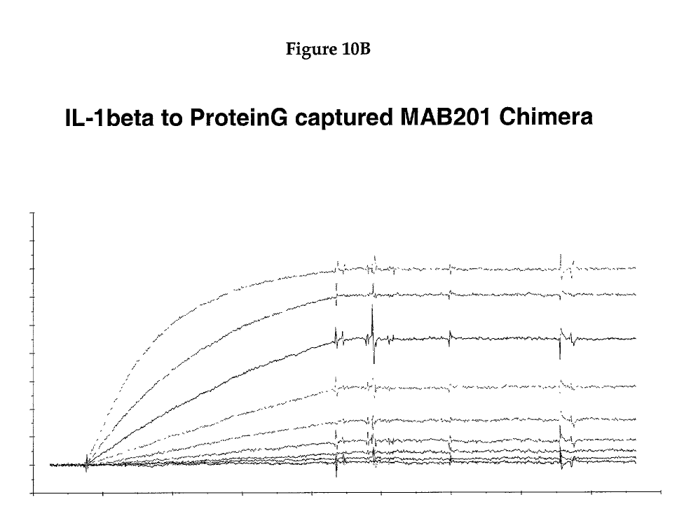

The purified antibodies were used for affinity measurements by BiaCore analysis. The purified chimeric and CDR grafted MAb201 were tested in a direct binding analysis on BIAcore. Briefly, various amount of IL-1β was injected over the protein G captured MAb201 surface. The binding affinity was estimated for the chimeric and the CDR grafted antibody at sub-nanomole with very slow off rates (FIGS. 10A and 10B).

Activity Assay

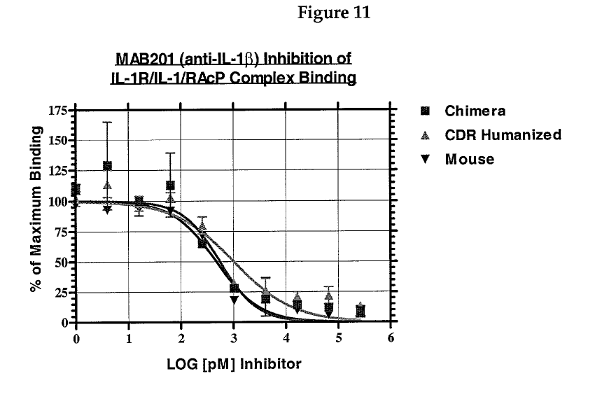

The activities of mouse, chimeric and humanized MAb201 in blocking formation of the IL-1β/IL-1 receptor/IL-1RacP complex are shown in FIG. 11. Varying concentrations of each antibody (262 nM to 1 pM) were incubated with biotinylated sIL-1 receptor (1 nM), IL-1β (1 nM), and ruthenium-tagged IL-1RacP (5 nM). After a 1-hour incubation, the resulting complexes were captured using magnetic streptavidin-coated beads. Competition of complex formation was evaluated using an Origen® Analyzer (IGEN Inc.), which measures an ECL signal proportional to the ruthenium tag captured in the complex. Data are expressed as a function of % maximum binding and are analyzed by non-linear regression, sigmoidal dose response (PRISM™) and are reported in Table 7 below.

TABLE 7
Activities of mouse, chimeric and humanized MAb201 in
blocking IL-1 β driven formation of the heterodimeric receptor
cpmlex of IL-1RI and IL-RAcP
Non-Linear Regression; Sigmoidal Dose Response (Variable Slope)

| Best-fit values | Chimera | CDR Humanized | Mouse |
|---|---|---|---|
| IC50 | 556 pM | 1023 pM | 476 pM |
| 95% Confidence | 220 to 1043 pM | 382 to 2738 pM | 275 to 824 pM |
| Intervals | | | |

What is claimed is:

1. A method of treating IL-1-mediated disease, which comprises administering an IL-1 selective binding agent that is capable of binding to IL-1β, wherein the selective binding agent-IL-1 complex is capable of binding to IL-1 receptor without activating said receptor.

2. The method of claim 1, wherein the selective binding agent-IL-1 complex is capable of blocking association of the IL-1 receptor with IL-1 receptor accessory protein.

3. The method of claim 1, wherein the IL-1 selective binding agent is an antibody.

4. The method of claim 3, wherein the antibody is a chimeric antibody.

5. The method of claim 3, wherein the antibody is a humanized antibody.

6. The method of claim 3, wherein the antibody is a fully human antibody.

7. The method of claim 3, wherein the antibody is generated by phage display.

8. The method of claim 3, wherein the antibody comprises SEQ ID NO: 2.

9. A method of blocking IL-1α and IL-1β from binding to the IL-1 receptor, which comprises administering an IL-1 selective binding agent that is capable of binding to IL-1β, wherein the selective binding agent-IL-1 complex is capable of binding to IL-1 receptor without activating said receptor.

10. A method of treating IL-1-mediated disease, which comprises administering an IL-1 selective binding agent that is capable of binding to IL-1α, wherein the selective binding agent-IL-1α complex is capable of binding to IL-1 receptor without activating said receptor.

11. The method of claim 10, wherein the selective binding agent-IL-1α complex is capable of blocking association of the IL-1 receptor with IL-1 receptor accessory protein.

12. The method of claim 10, wherein the IL-1 selective binding agent is an antibody.

13. The method of claim 12, wherein the antibody is a chimeric antibody.

14. The method of claim 12, wherein the antibody is a humanized antibody.

15. The method of claim 12, wherein the antibody is a fully human antibody.

16. The method of claim 12, wherein the antibody is generated by phage display.

17. A method of blocking IL-1α and IL-1β from binding to the IL-1 receptor, which comprises administering an IL-1 selective binding agent that is capable of binding to IL-1α, wherein the selective binding agent-IL-1α complex is capable of binding to IL-1 receptor without activating said receptor.
